(19) United States
(12) Patent Application Publication
(10) Pub. No.: US 2024/0400576 A1
(43) Pub. Date: Dec. 5, 2024

(54) NITROGEN-CONTAINING DERIVATIVES OF SALINOMYCIN, SYNTHESIS AND USES THEREOF

(71) Applicants: Centre National de la Recherche Scientifique, PARIS (FR); Institut Curie, PARIS CEDEX 05 (FR); Institut National de la Santé et de la Recherche Médicale (INSERM), PARIS (FR); Adam Mickiewicz University, POZNAN (FR)

(72) Inventors: Raphaël RODRIGUEZ, VERS-PONT-DU-GARD (FR); Dominika CZERWONKA, POZNAN (PL); Michal ANTOSZCZAK, BIEDRUSKO (PL); Adam HUCZYNSKI, POZNAN (PL)

(21) Appl. No.: 18/686,402

(22) PCT Filed: Sep. 2, 2022
(86) PCT No.: PCT/EP2022/074454

§ 371 Date: Feb. 23, 2024

(30) Foreign Application Priority Data
Sep. 3, 2021 (EP) 21306210.2

(51) Int. Cl. C07D 493/20; A61K 31/35; A61P 35/00

(52) U.S. Cl. CPC C07D 493/20; A61K 31/35; A61P 35/00

(57) ABSTRACT

The present invention relates to nitrogen-containing derivatives of salinomycin having the formula (I), as well as pharmaceutically acceptable salt thereof, and synthesis and uses thereof, in particular for the treatment and/or prevention of cancer including for preventing cancer metastasis and/or for preventing cancer recurrence and/or for decreasing resistance to a chemotherapy in a subject.

The present invention concerns novel nitrogen-containing derivatives of salinomycin, their synthesis and their uses in therapy, notably for the treatment of cancer.

Resistance to chemotherapy and radiotherapy remains a major obstacle in the successful treatment of cancer. Resistance may occur during cancer treatment because of many reasons, such as some of the cancer cells which are not killed can mutate and become resistant, gene amplification resulting in the overexpression of a protein that renders the treatment ineffective may occur, or cancer cells may develop a mechanism to inactivate the treatment.

Cancer stem cells (CSCs) have been shown to be refractory to conventional therapeutic agents, can promote metastasis, and have been linked to cancer relapse. Salinomycin can selectively kill CSCs. Salinomycin derivatives accumulate in lysosomes and sequester iron in this organelle. As a result, accumulation of iron leads to the production of reactive oxygen species (ROS) and lysosomal membrane permeabilization, which in turn promotes cell death by ferroptosis (a recently characterized cell death mechanism i.e., reliant on iron and mediated by ROS). Thus, iron homeostasis in CSCs is key. This mechanism creates opportunities toward the development of next-generation therapeutics.

Salinomycin is a potent antiproliferative drug in the treatment of drug-resistant cancer cell lines. However, this molecule includes a carboxyl radical which is responsible for its degradation in the cells.

Therefore, there is a need for the development of novel and efficient anti-cancer drugs, which would be efficient while being specific for tumoral tissues. Especially, there is a need for novel and efficient anti-cancer drugs able to kill both cancer stem cells and therapy-resistant cancer cells.

There is also a need for new molecules able to target the lysosomal iron, and also inducing the cell death of therapy-resistant cancer cells.

The aim of the present invention is thus to provide salinomycin derivatives able to target the lysosomal iron and also able to induce the cell death of therapy-resistant cancer cells. Another aim of the present invention is to provide salinomycin derivatives with anti-cancer activity, and preferably being able to kill both cancer stem cells and therapy-resistant cancer cells.

Therefore, the present invention relates to a compound having the formula (I):

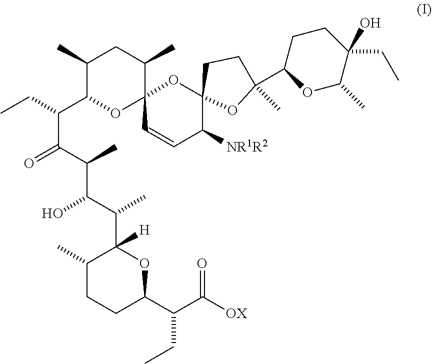

wherein:
R¹ and R² are identical or different and independently chosen from the group consisting of: H, a (C₁-C₂₀) alkyl group, a (C₃-C₆) cycloalkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: amino, hydroxyl, thiol, halogen, (C₁-C₆) alkyl, (C₁-C₆) alkoxy, (C₁-C₆) alkylthio, (C₁-C₆) alkylamino, halo (C₁-C₆) alkyl, carboxyl and carboxy (C₁-C₆) alkyl;
X is H, K or Na,
   with the proviso that at least one of R¹ and R² is other than H, and the following compound being excluded:

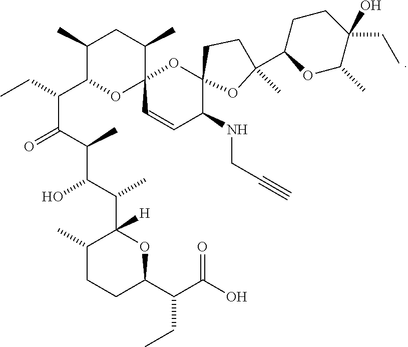 [SMILES: C#CCN[C@H]1C=C[C@]2(O[C@H]([C@@H](CC)C(=O)[C@@H](C)[C@@H](O)[C@H](C)[C@@H]3O[C@@H]([C@@H](CC)C(=O)O)CC[C@@H]3C)[C@@H](C)C[C@H]2C)O[C@@]12CC[C@@](C)([C@H]1CC[C@](O)(CC)[C@H](C)O1)O2]

The following definitions are set forth to illustrate and define the meaning and scope of the various terms used to describe the invention herein.

The expression “Cₜ-C_{z}” means a carbon-based chain which can have from t to z carbon atoms, for example C₁-C₃ means a carbon-based chain which can have from 1 to 3 carbon atoms. The term “alkyl group” means: a linear or branched, saturated, hydrocarbon-based aliphatic group comprising, unless otherwise mentioned, from 1 to 20 carbon atoms, preferably from 1 to 15, and more preferably from 3 to 10, or 1 to 6, carbon atoms. By way of examples, mention may be made of methyl, ethyl, n-propyl, isopropyl, n-butyl, isobutyl, tert-butyl, n-pentyl, isopentyl, n-hexyl, octyl or dodecyl groups, and preferably ethyl, n-propyl, n-butyl, isobutyl, n-pentyl, n-hexyl or dodecyl groups.

The term “aryl group” means: a cyclic aromatic group comprising between 6 and 10 carbon atoms. By way of examples of aryl groups, mention may be made of phenyl or naphthyl groups. The term “cycloalkyl group” means: a cyclic carbon-based group comprising, unless otherwise mentioned, from 3 to 6 carbon atoms. By way of examples, mention may be made of cyclopropyl, cyclobutyl, cyclopentyl, cyclohexyl, etc. groups. Preferably, the (C₃-C₆) cycloalkyl is cyclopropyl. The term “alkynyl” as employed herein includes unsaturated, nonaromatic, hydrocarbon groups having 2 to 20 carbons, preferably 2 to 6 carbons, and comprising at least one triple bond. Preferably, the alkyne group is linear. Preferably, the alkynyl group is a —(CH₂)ₙ—C≡CH group, n being an integer comprised from 1 to 4.

Preferably, the alkynyl group comprises 2 to 10 carbon atoms, preferably 2 to 6 carbon atoms. Preferably, the alkynyl group comprises 3 carbon atoms. Preferably, the alkynyl is propynyl. When an alkyl radical is substituted with an aryl group, the term “arylalkyl” or “aralkyl” radical is used. The “arylalkyl” or “aralkyl” radicals are aryl-alkyl-radicals, the aryl and alkyl groups being as defined above. Among the arylalkyl radicals, mention may in particular be made of the benzyl or phenethyl radicals.

Preferably, the arylalkyl according to the invention is a group —(CH₂)ₙ-aryl, wherein n is an integer from 1 to 5, and aryl is as defined above.

The aryl may be substituted by at least one substituent chosen from the group consisting of: amino (—NH₂), hydroxyl (—OH), thiol (—SH), halogen, (C₁-C₆) alkyl, (C₁-C₆) alkoxy, (C₁-C₆) alkylthio, (C₁-C₆) alkylamino, halo (C₁-C₆) alkyl, carboxyl and carboxy (C₁-C₆) alkyl.

Preferably, the aryl may be substituted by at least one substituent chosen from the group consisting of: a hydroxyl group, a (C₁-C₂₀) alkyl group, preferably a (C₁-C₆) alkyl group, and a halogen.

The term “halogen” means: a fluorine, a chlorine, a bromine or an iodine.

According to the invention, the term “alkoxy” means: an —O-alkyl radical where the alkyl group is as previously defined. By way of examples, mention may be made of —O-(C₁-C₄) alkyl groups, and in particular the —O-methyl group, the —O-ethyl group, the —O-propyl group, the —O-isopropyl group, the —O-butyl group, the —O-isobutyl group or the —O-tert-butyl group.

According to the invention, the term “alkylamino” means: an —NH-alkyl group, the alkyl group being as defined above.

According to the invention, the term “alkylthio” means: an —S-alkyl group, the alkyl group being as defined above.

According to the invention, the term “haloalkyl” means: an alkyl group as defined above, in which one or more of the hydrogen atoms is (are) replaced with a halogen atom. By way of example, mention may be made of fluoroalkyls, in particular CF₃ or CHF₂.

According to the present invention, the term “carboxyalkyl” means: an HOOC-alkyl group, the alkyl group being as defined above. As examples of carboxyalkyl groups, mention may in particular be made of carboxymethyl or carboxyethyl.

According to the present invention, the term “carboxyl” means: a COOH group.

Preferably, the aryl is substituted, preferably in para position, by at least one group chosen from a hydroxyl group, a (C₁-C₆) alkyl group such as a methyl, and a halogen such as F or C₁. Preferably, the aralkyl group is a benzyl which is not substituted, or substituted, preferably in para position, by at least one group chosen from a hydroxyl group, a (C₁-C₆) alkyl group such as a methyl, and a halogen such as F or C₁.

As mentioned above, R¹ and R² are not simultaneously H. Thus, when R¹ is H, then R² is other than H, and when R² is H, then R¹ is other than H.

According to an embodiment, in formula (I), R¹ and R² are different.

According to an embodiment, in formula (I), R¹ is selected from the group consisting of: a (C₁-C₂₀) alkyl group, a (C₃-C₆) cycloalkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, hydroxyl, amino, thiol, (C₁-C₆) alkoxy, (C₁-C₆) alkylthio, (C₁-C₆) alkylamino, halo (C₁-C₆) alkyl, carboxyl and carboxy (C₁-C₆) alkyl, and R² is H.

According to an embodiment, in formula (I), R¹ is selected from the group consisting of: a (C₁-C₂₀) alkyl group, a (C₃-C₆) cycloalkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, and hydroxyl, and R² is H.

Thus, a preferred group of compounds according to the invention consists of compounds having the formula (II):

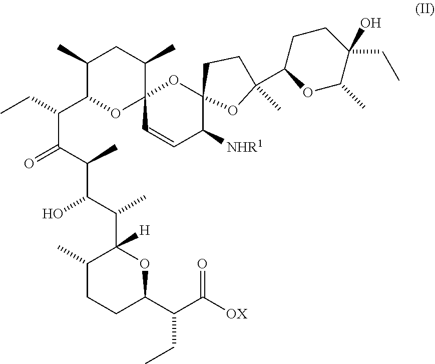

R¹ and X being as defined above.

According to an embodiment, in formula (I) or (II), X is H.

According to an embodiment, in formula (I) or (II), X is Na.

According to an embodiment, in formula (I), R¹ is selected from the group consisting of: a (C₁-C₂₀) alkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, and hydroxyl, and R² is H.

Preferably, in formula (II), R¹ is selected from the group consisting of: a (C₁-C₂₀) alkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, and hydroxyl.

According to an embodiment, in formula (I), R¹ is a linear (C₂-C₁₂) alkyl group, a propyn-1-yl group, or a benzyl group, optionally substituted with hydroxyl, methyl or halogen, preferably in para position, and R² is H.

Preferably, in formula (II), R¹ is a linear (C₂-C₁₂) alkyl group, a propyn-1-yl group, or a benzyl group, optionally substituted with hydroxyl, methyl or halogen, preferably in para position.

According to an embodiment, in formula (I), R¹ and R² are identical.

According to this embodiment, R¹ and R² are chosen from the group consisting of: a (C₁-C₂₀) alkyl group, preferably a linear (C₂-C₁₂) alkyl group, and a (C₂-C₂₀) alkynyl group, preferably a propynyl group.

Thus, a preferred group of compounds according to the invention consists of compounds having the formula (III):

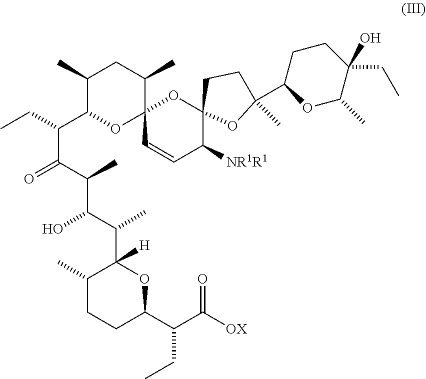

R¹ and X being as defined above.

According to an embodiment, in formula (III), X is H.

According to an embodiment, in formula (III), X is Na.

Preferably, in formula (III), R¹ is chosen from the group consisting of: a (C₁-C₂₀) alkyl group, preferably a linear (C₂-C₁₂) alkyl group, and a (C₂-C₂₀) alkynyl group, preferably a propynyl group.

As preferred compounds, the followings may be mentioned:

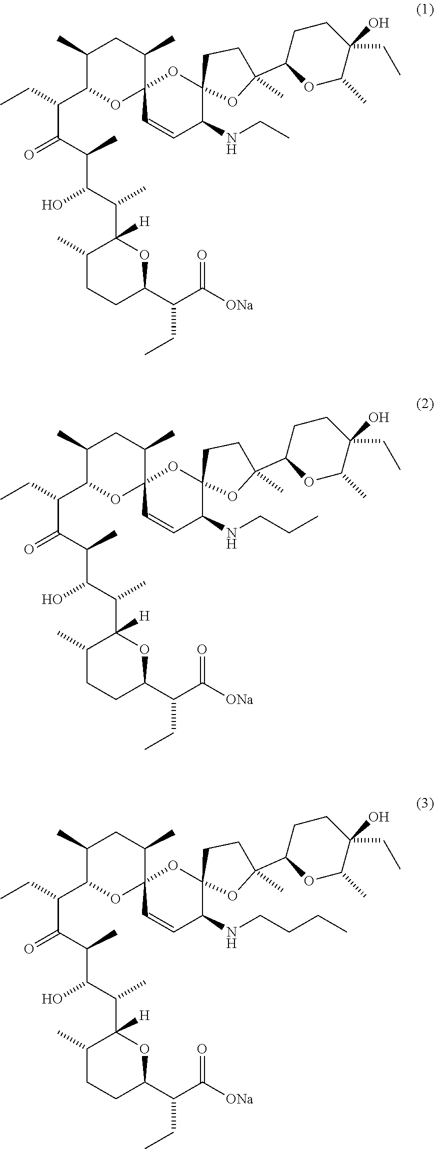

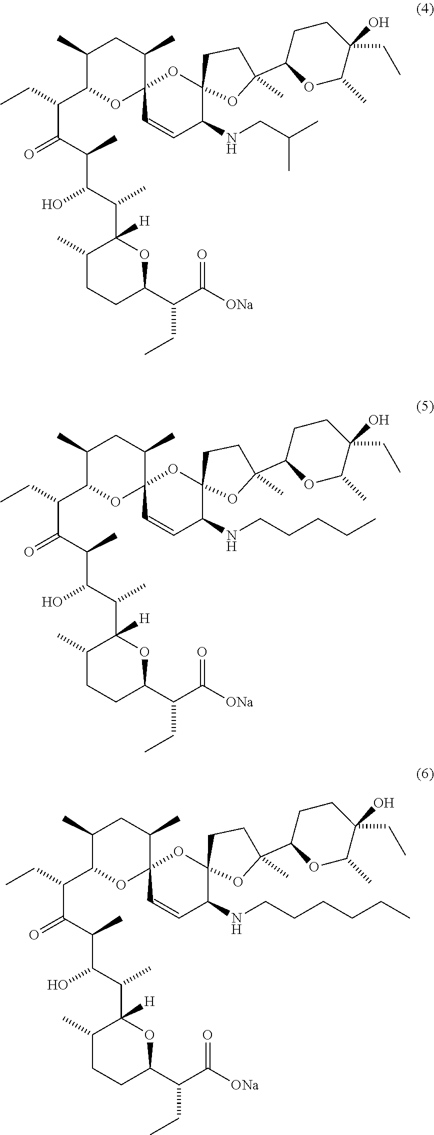

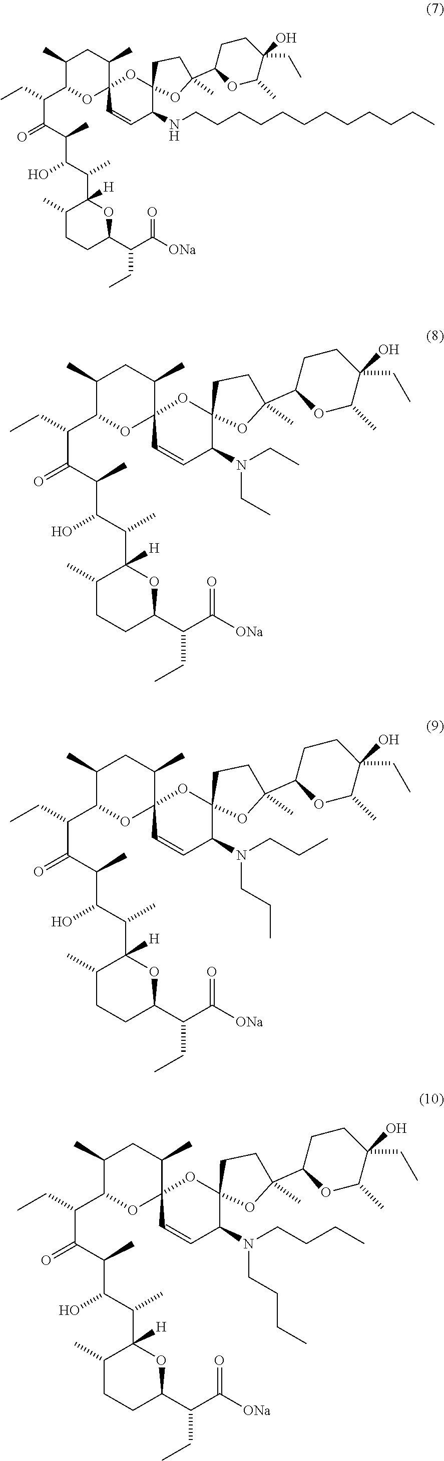

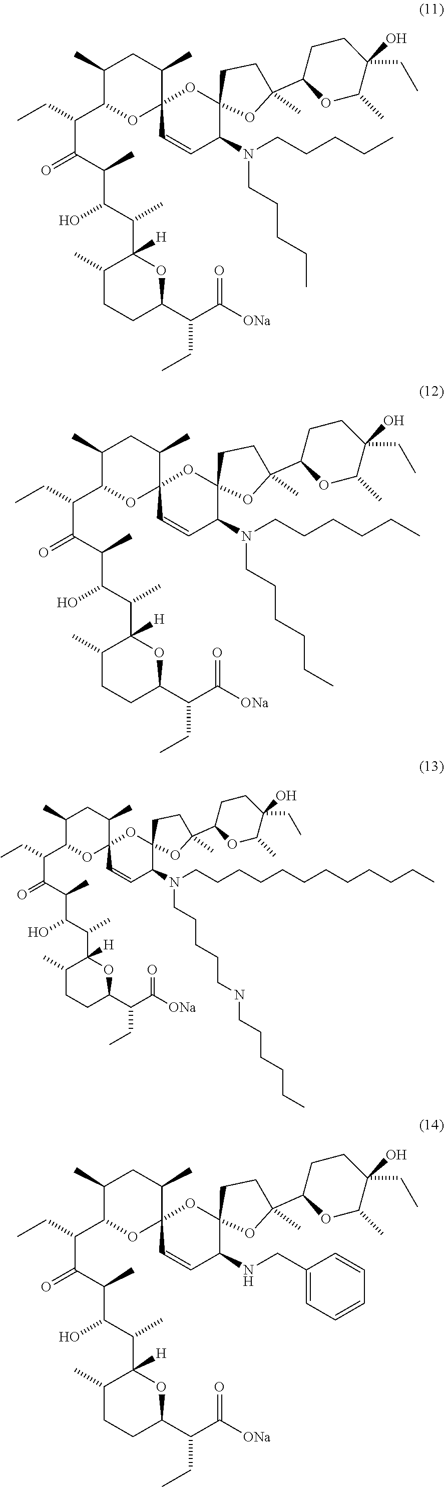

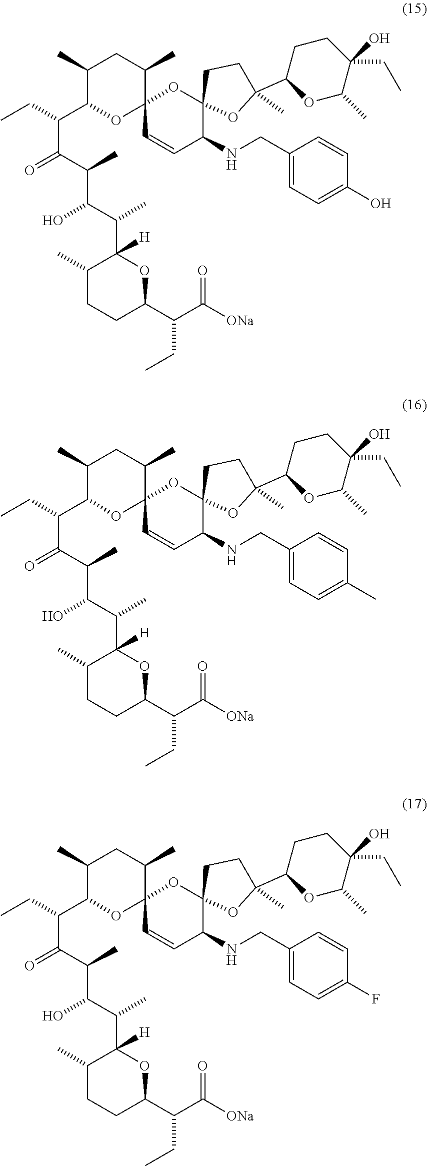

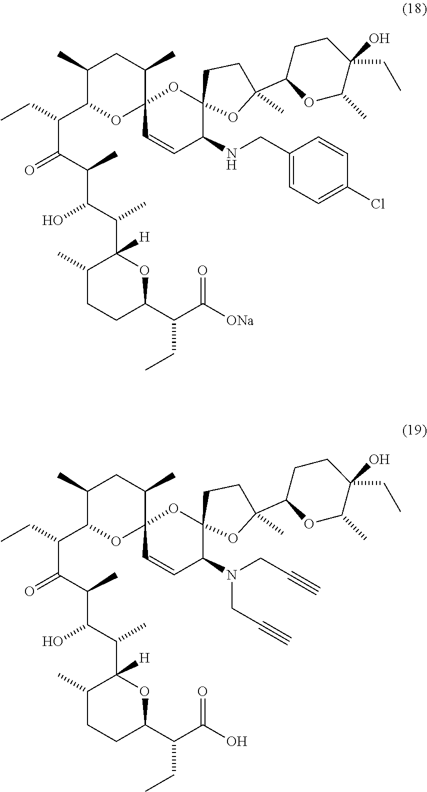

As shown in the examples, the compounds of formula (I) of the invention are very specific for cancerous cell lines of various cancers as compared to non-cancerous cell lines.

Moreover, as shown in Table 1, the compounds of formula (I) of the invention show an anticancer activity which is selective.

### Preparation of the Compounds of the Invention

The compounds of the invention may be prepared by the following process, which is illustrated in the examples.

The compounds of formula (I) are prepared from the compound C₂₀-epi-aminosalinomycin or its sodium salt.

The present invention thus also relates to a process for the preparation of a compound of formula (II) or (III) as defined above, comprising the following steps:
the addition of an aldehyde R¹CHO, R¹ being as defined above, to a solution of a sodium salt of C20-epi-aminosalinomycin in a solvent, in order to obtain the corresponding imine, followed by the reduction of said imine into the corresponding amine compound of formula (II) or (III), or
the reaction of a bromide compound R¹Br, R¹ being as defined above, with the compound C20-epi-aminosalinomycin, preferably in a solvent.

The C20-epi-aminosalinomycin has the following formula:

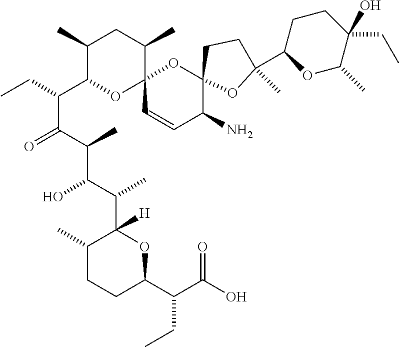 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@H](N)[C@]3(CC[C@@](C)([C@H]4CC[C@](O)(CC)[C@H](C)O4)O3)O2)[C@H](C)C[C@@H]1C]

For example, an aldehyde R¹CHO is added to a solution of a sodium salt of C20-epi-aminosalinomycin in a solvent such as dichloromethane. The solution is then preferably stirred for 24 h at room temperature, and after that time a solution of NaBH₃CN in methanol was added dropwise. The reaction mixture was further stirred and then the solvent was evaporated under reduced pressure.

For example, when R¹=R²=alkynyl, the compound C20-epi-aminosalinomycin is reacted with a bromide compound being R¹Br such as propargyl bromide, in a solvent, such as acetonitrile, preferably at 60° C.

### Compositions and Uses

The present invention also relates to the compound as defined above, in particular having the formula (I) or (II), for use as drug.

The present invention also relates to a medicament comprising a compound as defined above, in particular having the formula (I) or (II), or a pharmaceutically acceptable salt thereof.

The present invention also relates to a pharmaceutical composition comprising, at least one compound as defined above, in particular having the formula (I) or (II), or a pharmaceutically acceptable salt thereof, and also at least one pharmaceutically acceptable excipient.

### Anti-Cancer Uses

The compounds of formula (I) of the invention may be used for preventing and/or treating cancer. By “preventing”, it is meant avoiding the cancer to occur.

By “treatment”, it is meant the curative treatment of cancer. A curative treatment is defined as a treatment that completely treat (cure) or partially treat cancer (i.e. induces tumor growth stabilization, retardation or regression).

The “subject” refers to any subject and typically designates a patient, preferably a subject undergoing a treatment of cancer such as immunotherapy, chemotherapy and/or radiotherapy. In any case, the subject is preferably a vertebrate, more preferably a mammal, even more preferably a human being.

By “cancer”, it is meant any type of cancer. The cancer may be solid or non-solid, and may be for example selected from a colon cancer, a colorectal cancer such as colorectal cancers with a BRAF mutation (especially BRAF V600E), a melanoma, a bone cancer, a breast cancer such as triple-negative breast cancer (i.e. breast cancer that tests negative for estrogen receptors, progesterone receptors, and excess HER2 protein), a thyroid cancer, a prostate cancer, an ovarian cancer, a lung cancer, a pancreatic cancer, a glioma such as a glioblastoma, a cervical cancer, an endometrial cancer, a head and neck cancer, a liver cancer, a bladder cancer, a renal cancer, a skin cancer, a stomach cancer, a testis cancer, an urothelial cancer or an adrenocortical carcinoma, leukemia such as acute myeloid leukemia, but also non-solid cancers such as lymphoma or multiple myeloma.

Preferably, the cancer is a colon cancer, a colorectal cancer such as colorectal cancers with a BRAF mutation (especially BRAF V600E), a breast cancer such as triple-negative breast cancer (i.e. breast cancer that tests negative for estrogen receptors, progesterone receptors, and excess HER2 protein), a pancreatic cancer, a glioma such as a glioblastoma, leukemia such as acute myeloid leukemia, lymphoma or multiple myeloma.

The cancer can be a metastatic cancer or not. A typical cancer is a cancer resistant to the first-line chemotherapy.

The present invention thus relates to a compound as defined above, in particular having the formula (I), (II) or (III), or a pharmaceutically acceptable salt thereof, for use for preventing and/or treating cancer.

Preferably, the cancer is selected from the group consisting of: a colon cancer, a colorectal cancer, a melanoma, a bone cancer, a breast cancer, a thyroid cancer, a prostate cancer, an ovarian cancer, a lung cancer, a pancreatic cancer, a glioma, a cervical cancer, an endometrial cancer, a head and neck cancer, a liver cancer, a bladder cancer, a renal cancer, a skin cancer, a stomach cancer, a testis cancer, an urothelial cancer or an adrenocortical carcinoma, leukemia, lymphoma, and multiple myeloma.

The invention also relates to the use of at least one compound of formula (I) for increasing the sensitivity of a cancer to a chemotherapeutic drug.

A further object of the invention is the use of at least one compound of formula (I) for decreasing the resistance of a cancer with respect to a chemotherapeutic drug.

The invention also relates to a product comprising:
a) at least one compound of formula (I) of the invention, and
b) at least one additional therapy,
as a combination product for a simultaneous, separate or sequential use for treating cancer, and/or for preventing cancer metastasis, and/or for preventing cancer recurrence, and/or for decreasing resistance to the additional therapy b), in a subject.

It also relates to the use of at least one compound of formula (I) of the invention, for preventing and/or treating a cancer in combination or in association with at least one additional therapy.

It further relates to the use of at least one compound of formula (I) of the invention, for preventing and/or treating a cancer in a subject treated by at least one additional therapy. The invention also relates to at least one compound of formula (I) of the invention, for use as an adjuvant cancer therapy. An adjuvant therapy is a therapy for treating cancer that is given besides a primary or initial therapy (“first-line therapy”), to maximize its effectiveness.

Said additional therapy b) may be immunotherapy, chemotherapy and/or radiotherapy. Preferably the additional therapy b) is immunotherapy and/or chemotherapy.

By “immunotherapy”, it is meant a therapy with is able to induce, enhance or suppress an immune response. Said immunotherapy is preferably chosen from cytokines, chemokines, growth factors, growth inhibitory factors, hormones, soluble receptors, decoy receptors; monoclonal or polyclonal antibodies, mono-specific, bi-specific or multi-specific antibodies, monobodies, polybodies; vaccination; or adoptive specific immunotherapy.

Preferably the immunotherapy is chosen from monoclonal or polyclonal antibodies, mono-specific, bi-specific or multi-specific antibodies, monobodies, polybodies, such as anti-angiogenic agents like Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (soluble hybrid receptor VEGF-A, PIGF (placenta growth factor) Aventis/Regeneron).

Preferably the immunotherapy is a monoclonal antibody, preferably an anti-checkpoint antibody. The anti-checkpoint antibodies comprise antibodies directed against an immune checkpoint, which may be chosen from PD1, PDL1, PDL2, CTLA4, BTLA, CD27, CD40, OX40, GITR (also called “Tumor necrosis factor receptor superfamily member 18” or TNFRSF18), CD137 (also called 4-1BB or TNFRS9), CD28, ICOS, IDO (indoleamine 2,3-dioxygenase), B7H3 (also called CD276), KIR2DL2 (also called killer cell immunoglobulin-like receptor 2DL2), NKG2 (a family of the C-type lectin receptors), LAG3 (also called Lymphocyte Activation Gene-3) and CD70. Preferably the anti-checkpoint antibodies are anti-PD1, anti-PDL1, anti-PDL2 or anti-CTLA4 antibodies. Anti-PD1 antibodies include nivolumab and pembrolizumab. Anti-CTLA4 antibodies include ipilimumab and tremelimumab.

The “chemotherapy” or “chemotherapeutic agent” refers to compounds which are used in the treatment of cancer and that have the functional property of inhibiting a development or progression of a neoplasm in a human, particularly a malignant (cancerous) lesion.

Chemotherapeutic agents have different modes of actions, for example, by influencing either DNA or RNA and interfering with cell cycle replication.

Examples of chemotherapeutic agents that act at the DNA level or on the RNA level are:
anti-metabolites, such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluorouracil and hydroxyurea;
alkylating agents, such as Melphalan, Busulfan, Cisplatin, Carboplatin, Cyclophosphamide, Ifosphamide, Dacarabazine, Fotemustine, Procarbazine, Chlorambucil, Thiotepa, Lomustine, Temozolomide;
anti-mitotic agents, such as Vinorelbine, Vincristine, Vinblastine, Docetaxel, Paclitaxel;
topoisomerase inhibitors, such as Doxorubicin, Amsacrine, Irinotecan, Daunorubicin, Epirubicin, Mitomycin, Mitoxantrone, Idarubicin, Teniposide, Etoposide, Topotecan;
antibiotics, such as actinomycin and bleomycin;
asparaginase;
anthracyclines or taxanes.

Other chemotherapeutic agents are tyrosine kinase inhibitors (TKIs). A number of TKIs are in late and early stage development for treatment of various types of cancer. Exemplary TKIs include, but are not limited to: BAY 43-9006 (Sorafenib, Nexavar®) and SU11248 (Sunitinib, Sutent®), Imatinib mesylate (Gleevec®, Novartis); Gefitinib (Iressa®, AstraZeneca); Erlotinib hydrochloride (Tarceva®, Genentech); Vandetanib (Zactima®, AstraZeneca), Tipifarnib (Zarnestra®, Janssen-Cilag); Dasatinib (Sprycel®, Bristol Myers Squibb); Lonafarnib (Sarasar®, Schering Plough); Vatalanib succinate (Novartis, Schering AG); Lapatinib (Tykerb®, GlaxoSmithKline); Nilotinib (Novartis); Lestaurtinib (Cephalon); Pazopanib hydrochloride (GlaxoSmithKline); Axitinib (Pfizer); Canertinib dihydrochloride (Pfizer); Pelitinib (National Cancer Institute, Wyeth); Tandutinib (Millennium); Bosutinib (Wyeth); Semaxanib (Sugen, Taiho); AZD-2171 (AstraZeneca); VX-680 (Merck, Vertex); EXEL-0999 (Exelixis); ARRY-142886 (Array BioPharma, AstraZeneca); PD-0325901 (Pfizer); AMG-706 (Amgen); BIBF-1120 (Boehringer Ingelheim); SU-6668 (Taiho); CP-547632 (OSI); (AEE-788 (Novartis); BMS-582664 (Bristol-Myers Squibb); JNK-401 (Celgene); R-788 (Rigel); AZD-1152 HQPA (AstraZeneca); NM-3 (Genzyme Oncology); CP-868596 (Pfizer); BMS-599626 (Bristol-Myers Squibb); PTC-299 (PTC Therapeutics); ABT-869 (Abbott); EXEL-2880 (Exelixis); AG-024322 (Pfizer); XL-820 (Exelixis); OSI-930 (OSI); XL-184 (Exelixis); KRN-951 (Kirin Brewery); CP-724714 (OSI); E-7080 (Eisai); HKI-272 (Wyeth); CHIR-258 (Chiron); ZK-304709 (Schering AG); EXEL-7647 (Exelixis); BAY-57-9352 (Bayer); BIBW-2992 (Boehringer Ingelheim); AV-412 (AVEO); YN-968D1 (Advenchen Laboratories); Staurosporin, Midostaurin (PKC412, Novartis); Perifosine (AEterna Zentaris, Keryx, National Cancer Institute); AG-024322 (Pfizer); AZD-1152 (AstraZeneca); ON-01910Na (Onconova); and AZD-0530 (AstraZeneca).

Herein described are also (i) a method for preventing or treating cancer, (ii) a method for increasing the sensitivity of a cancer to a chemotherapeutic agent, and (iii) a method for decreasing the resistance of a cancer with respect to a chemotherapeutic drug, each of said methods comprising administering to a subject in need thereof with an effective amount of at least one compound of formula (I) as defined above, preferably together with a chemotherapeutic drug.

The compound of formula (I) of the invention is preferably administered at a therapeutically effective amount or dose. As used herein, “a therapeutically effective amount or dose” refers to an amount of the compound of the invention which prevents, removes, slows down the disease, or reduces or delays one or several symptoms or disorders caused by or associated with said disease in the subject, preferably a human being. The effective amount, and more generally the dosage regimen, of the compound of the invention and pharmaceutical compositions thereof may be determined and adapted by the one skilled in the art. An effective dose can be determined by the use of conventional techniques and by observing results obtained under analogous circumstances. The therapeutically effective dose of the compound of the invention will vary depending on the disease to be treated or prevented, its gravity, the route of administration, any co-therapy involved, the patient's age, weight, general medical condition, medical history, etc. Typically, the amount of the compound to be administered to a patient may range from about 0.01 to 500 mg/kg of body weight for a human patient. In a particular embodiment, the pharmaceutical composition according to the invention comprises 0.01 mg/kg to 300 mg/kg of the compound of the invention, preferably from 0.01 mg/kg to 3 mg/kg, for instance from 25 to 300 mg/kg.

In a particular aspect, the compounds of the invention can be administered to the subject by parenteral route, topical route, oral route or intravenous injection. The compound or the nanoparticle of the invention may be administered to the subject daily (for example 1, 2, 3, 4, 5, 6 or 7 times a day) during several consecutive days, for example during 2 to 10 consecutive days, preferably from 3 to 6 consecutive days. Said treatment may be repeated during 1, 2, 3, 4, 5, 6 or 7 weeks, or every two or three weeks or every one, two or three months. Alternatively, several treatment cycles can be performed, optionally with a break period between two treatment cycles, for instance of 1, 2, 3, 4 or 5 weeks. The compound of the invention can for example be administered as a single dose once a week, once every two weeks, or once a month. The treatment may be repeated one or several times per year.

Doses are administered at appropriate intervals which can be determined by the skilled person. The amount chosen will depend on multiple factors, including the route of administration, duration of administration, time of administration, the elimination rate of the compound, or of the various products used in combination with said compound, the age, weight and physical condition of the patient and his/her medical history, and any other information known in medicine.

The administration route can be oral, topical or parenteral, typically rectal, sublingual, intranasal, intra-peritoneal (IP), intra-venous (IV), intra-arterial (IA), intra-muscular (IM), intra-cerebellar, intrathecal, intratumoral and/or intradermal. The pharmaceutical composition is adapted for one or several of the above-mentioned routes. The pharmaceutical composition is preferably administered by injection or by intravenous infusion of suitable sterile solutions, or in the form of liquid or solid doses via the alimentary canal.

The present invention also relates to a composition comprising, in a pharmaceutically acceptable medium, at least one compound of formula (I) according to the invention. Such a composition comprises a pharmaceutically acceptable medium (or carrier).

The carrier must be “acceptable” in the sense of being compatible with the other ingredients of the formulations and not deleterious to the recipient thereof.

The pharmaceutical composition can be formulated as solutions in pharmaceutically compatible solvents or as gels, oils, emulsions, suspensions, or dispersions in suitable pharmaceutical solvents or vehicles, or as pills, tablets, capsules, powders, suppositories, etc. that contain solid vehicles in a way known in the art, possibly through dosage forms or devices providing sustained and/or delayed release. For this type of formulation, an agent such as cellulose, lipids, carbonates or starches are used advantageously.

Agents or vehicles that can be used in the formulations (liquid and/or injectable and/or solid) are excipients or inert vehicles, i.e. pharmaceutically inactive and non-toxic vehicles.

Mention may be made, for example, of saline, physiological, isotonic and/or buffered solutions, compatible with pharmaceutical use and known to those skilled in the art. The compositions may contain one or more agents or vehicles chosen from dispersants, solubilizers, stabilizers, preservatives, etc.

Particular examples are methylcellulose, hydroxymethylcellulose, carboxymethylcellulose, cyclodextrins, polysorbate 80, mannitol, gelatin, lactose, liposomes, vegetable oils or animal, acacia, etc. Preferably, vegetable oils are used.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations suitable for parenteral administration conveniently comprise a sterile oily or aqueous preparation of the active ingredient which is preferably isotonic with the blood of the recipient. Every such formulation can also contain other pharmaceutically compatible and non-toxic auxiliary agents, such as, e.g. stabilizers, antioxidants, binders, dyes, emulsifiers or flavoring substances.

The invention is now illustrated by the following examples.

This research was by funded by the European Research Council under the European Union's Horizon 2020 research and innovation programme grant agreement No 647973, the Foundation Charles Defforey-Institut de France, Ligue Contre le Cancer (Equipes Labellisées).

### EXAMPLES

### Example 1: Preparation of Mono- and Di-Substituted C₂₀-Epi-Amine Analogues of Salinomycin

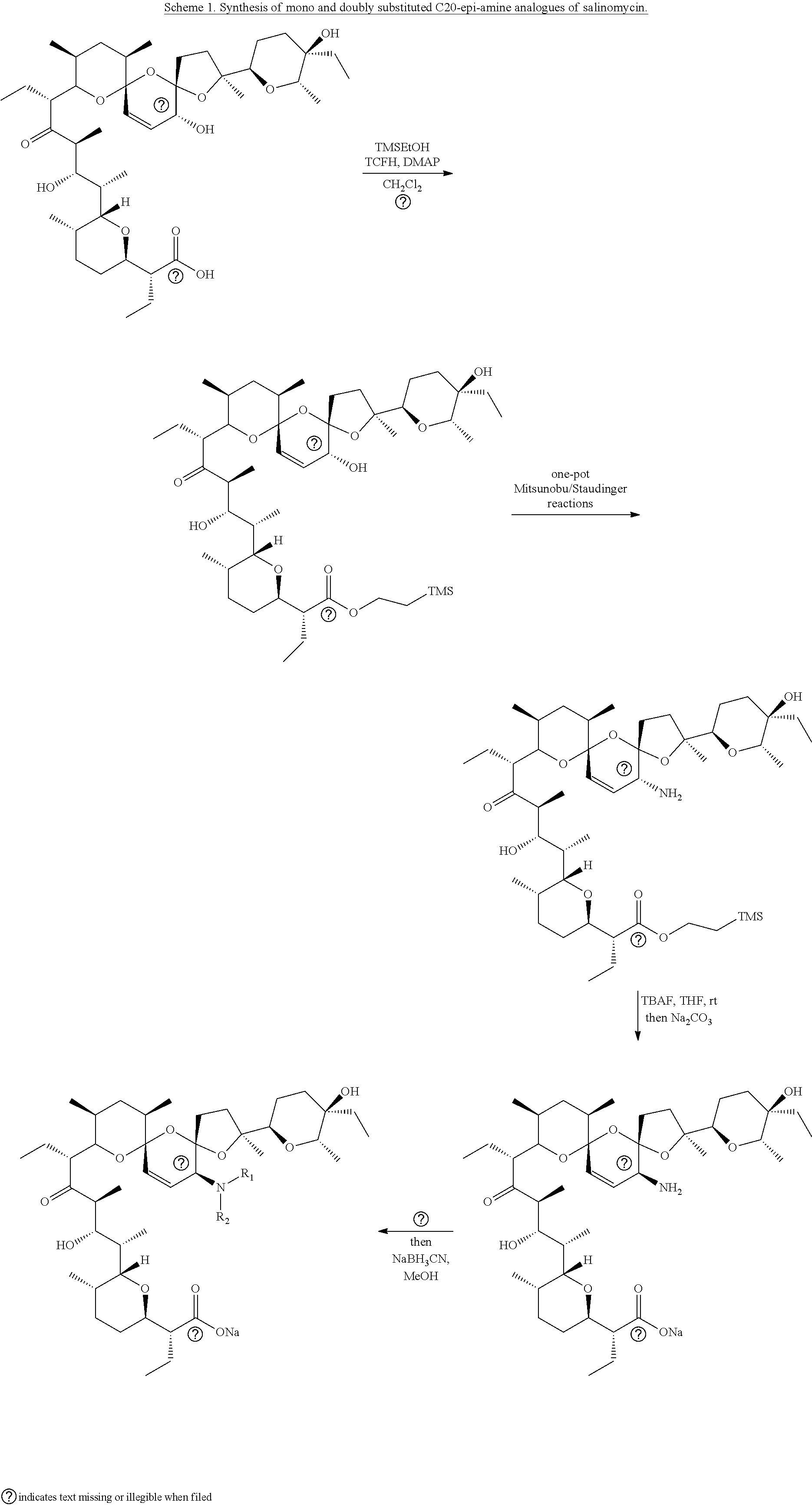

### General Procedure for the Preparation of Sodium Salts of C₂₀-Epi-Aminosalinomycin Analogues

### Step 1. Blocking of the C₁ Functionality

To a stirring solution of salinomycin (3.00 g, 1.0 equiv.) in 50 mL of CH₂Cl₂ in an ice bath was added: DMAP (2.38 g, 5.0 equiv.), TMSEtOH (2.77 g, 6.0 equiv.) and TCFH (1.31 g, 1.2 equiv.). The resulting mixture was stirred at room temperature overnight. The reaction mixture was then concentrated under reduced pressure. Purification on silica gel using the CombiFlash system (0-+40% EtOAc/n-hexane) gave the product of the reaction as yellow oil (1.70 g, 50% yield).

### Step 2. One-Pot Mitsunobu/Staudinger Reactions

To a solution of salinomycin-EtTMS ester (1.70 g, 1.0 equiv.) in 20 mL of anhydrous THF in an ice bath was added PPh3 (784 mg, 1.5 equiv.). After 20 minutes, DIAD (481 mg, 1.2 equiv.) was slowly added to a mixture, followed by the addition of DPPA (605 mg, 1.1 equiv.). The solution was stirred at room temperature for 24 h. After this time, PPh3 (1.57 g, 3.0 equiv.) was added at one portion, followed by the addition of 0.5 ml of water. The mixture was stirred at room temperature for next 24 h, and the reaction progress was monitored by TLC. The reaction mixture was then concentrated under reduced pressure. Purification on silica gel using the CombiFlash system (0-+50% acetone/CHCI3) gave the product of the reaction as yellow oil (716 mg, 42% yield).

### Step 3. Unblocking of the C1 Functionality

The C1-EtTMS ester of C20-epi-aminosalinomycin (716 mg, 1.0 equiv.) was dissolved in 15 mL of anhydrous THF at room temperature. The 1.0 M solution of TBAF in THF (2.53 mL, 3.0 equiv.) was then added dropwise. The solution was left to stir at room temperature for next 24 h. The reaction mixture was then concentrated under reduced pressure. Purification on silica gel using the CombiFlash system (0-+50% acetone/CHCl₃) gave the product of the reaction as yellow oil. The product was then dissolved in CH₂Cl₂ and washed with 0.1 M solution of Na₂CO₃. The separated organic layers were concentrated under reduced pressure. The residue was then evaporated several times with n-pentane to give quantitatively the sodium salt of C20-epi-aminosalinomycin as a white amorphous solid (648 mg).

### General Procedure for the Preparation of C20-Epi-Amine Analogues of Salinomycin (Compounds of Formula (I) Wherein X=Na)

To a stirred solution of sodium salt of C20-epi-aminosalinomycin (100 mg, 0.13 mmol, 1.0 equiv.) in 5 mL of CH₂Cl₂, the corresponding aldehyde was added (1.0 equiv. for mono substituted analogues or 5.0 equiv. for doubly substituted analogues). The solution was stirred at room temperature for 24 h, and after that time the solution of NaBH₃CN (10 mg, 0.16 mmol, 1.2 equiv.) in 2 mL of MeOH was added dropwise. The reaction mixture was stirred for additional 30 minutes. After that, the solvent was evaporated under reduced pressure. Purification of the filtrated residue on silica gel using the CombiFlash system gave the corresponding products of the reactions as the white amorphous solids.

### Example 1.1: Preparation of Compound (1)

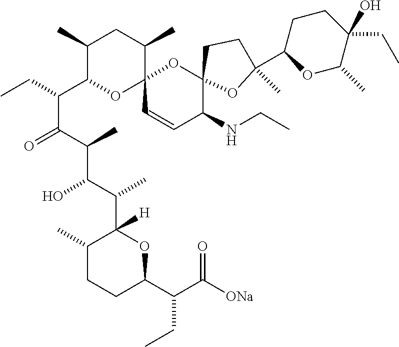

Yield: 35 mg, 70%. Isolated as a white amorphous solid, >95% pure by NMR and a single spot by TLC.

R_{f}: 0.57 in 60% acetone/CHCl₃. Strains green with PMA.

¹H NMR (400 MHZ, CD₂Cl₂) δ 6.31 (dd, J=10.7, 5.6 Hz, ¹H), 6.12 (d, J=10.8 Hz, ¹H), 4.29 (q, J=6.8 Hz, ¹H), 4.12 (d, J=10.4 Hz, ¹H), 3.77 (dt, J=13.8, 6.9 Hz, ¹H), 3.64 (dd, J=10.1, 2.1 Hz, ¹H), 3.60 (d, J=10.2 Hz, ¹H), 3.37 (dd, J=11.9, 2.1 Hz, ¹H), 2.86 (d, J=5.6 Hz, ¹H), 2.81-2.73 (m, 2H), 2.72-2.68 (m, ¹H), 2.67-2.57 (m, 2H), 2.00-0.50 (m, 60H) ppm.

¹³C NMR (101 MHZ, CD₂Cl₂) δ 218.4, 184.1, 129.9, 122.7, 110.1, 99.7, 89.2, 76.4, 76.3, 75.5, 75.1, 71.8, 70.2, 67.7, 56.1, 55.5, 51.6, 50.9, 42.8, 40.7, 39.5, 37.5, 36.5, 33.4, 33.1, 33.0, 29.8, 28.6, 28.3, 27.4, 24.2, 21.4, 20.6, 17.9, 17.3, 16.5, 16.4, 15.1, 13.5, 12.8, 12.4, 11.0, 7.0, 6.8 ppm.

FT-IR (KBr): 3319 (br, m), 2963 (s), 2935 (s), 2875 (m), 1714 (s), 1568 (s), 1460 (s), 1407 (m) cm⁻¹.

HRMS-ESI (m/z): [M+H]⁺ Calcd for C44H₇₅NO₁₀Na⁺880.5283; Found 880.5270; [M−Na⁺2H]⁺ Calcd for C₄₄H₇₆NO₁₀⁺778.5464; Found 778.5460.

### Example 1.2: Preparation of Compound (2)

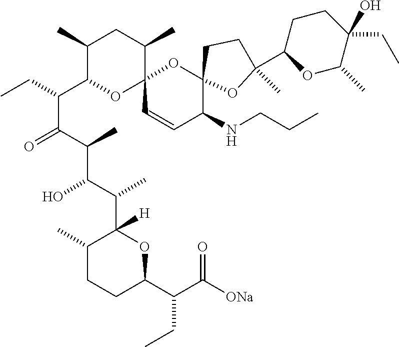

Yield: 35 mg, 66%. Isolated as a white amorphous solid, >95% pure by NMR and a single spot by TLC.

R_{f}: 0.23 in 66% EtOAc/n-hexane. Strains green with PMA.

¹H NMR (400 MHZ, CD₂Cl₂) δ 6.27 (dd, J=10.7, 5.6 Hz, ¹H), 6.08 (d, J=10.8 Hz, ¹H), 4.28 (q, J=6.7 Hz, ¹H), 4.11 (d, J=10.2 Hz, ¹H), 3.78 (dd, J=11.1, 4.8 Hz, ¹H), 3.64 (dd, J=16.6, 6.3 Hz, 2H), 2.97-2.87 (m, 2H), 2.79-2.68 (m, 2H), 2.68-2.61 (m, 2H), 2.11-2.04 (m, 2H), 2.00-0.50 (m, 61H) ppm.

¹³C NMR (101 MHZ, CD₂Cl₂) δ 218.6, 184.1, 130.3, 122.0, 110.4, 99.6, 89.22, 76.5, 76.4, 75.4, 75.1, 71.8, 70.2, 67.8, 56.1, 53.3, 51.6, 50.9, 47.6, 40.7, 39.5, 37.7, 36.5, 33.3, 33.2, 33.0, 30.0, 28.6, 28.2, 27.5, 24.8, 24.1, 23.2, 21.3, 20.6, 17.9, 17.4, 16.6, 15.1, 13.5, 12.8, 12.4, 11.0, 7.1, 6.8 ppm.

FT-IR (KBr): 3289 (br, m), 2962 (s), 2933 (s), 2874 (m), 1713 (s), 1660 (w), 1568 (s), 1459 (s), 1407 (m) cm-1.

HRMS-ESI (m/z): [M+H]⁺ Calcd for C₄₅H₇₇NO₁₀Na⁺814.5440; Found 814.5426; [M−Na⁺2H]⁺ Calcd for C45H₇₈NO₁₀⁺792.5620; Found 792.5619.

### Example 1.3: Preparation of Compound (3)

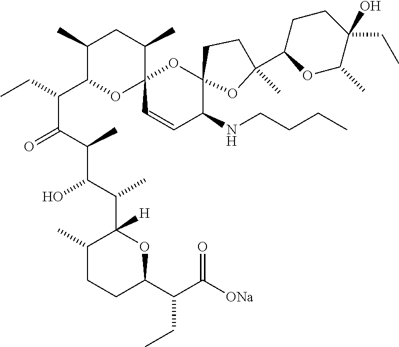

Yield: 36 mg, 68%. Isolated as a white amorphous solid, >95% pure by NMR and a single spot by TLC.

R_{f}: 0.29 in 66% EtOAc/n-hexane. Strains green with PMA.

¹H NMR (400 MHZ, CD₂Cl₂) δ 6.31 (dd, J=10.7, 5.6 Hz, ¹H), 6.11 (d, J=10.8 Hz, ¹H), 4.29 (q, J=6.7 Hz, ¹H), 4.12 (d, J=10.4 Hz, ¹H), 3.78 (dd, J=11.1, 4.8 Hz, ¹H), 3.64 (dd, J=9.9, 1.8 Hz, ¹H), 3.60 (d, J=10.2 Hz, ¹H), 3.37 (dd, J=11.9, 2.1 Hz, ¹H), 2.84 (d, J=5.6 Hz, ¹H), 2.80-2.69 (m, 3H), 2.68-2.62 (m, ¹H), 2.60-2.52 (m, ¹H), 2.00-0.50 (m, 64H) ppm.

¹³C NMR (101 MHZ, CD₂Cl₂) δ 218.4, 184.1, 130.0, 122.6, 110.1, 99.7, 89.2, 76.5, 76.3, 75.5, 75.1, 71.8, 70.2, 67.7, 56.1, 55.8, 51.6, 50.9, 48.27, 40.7, 39.4, 37.5, 36.5, 33.5, 33.4, 33.2, 33.0, 29.8, 28.6, 28.3, 27.4, 24.2, 21.4, 20.8, 20.6, 17.9, 17.3, 16.5, 15.1, 14.3, 13.5, 12.8, 12.4, 11.0, 7.0, 6.8 ppm.

FT-IR (KBr): 3288 (br, m), 2960 (s), 2931 (s), 2873 (m), 1713 (s), 1661 (w), 1567 (s), 1459 (s), 1406 (m) cm-1.

HRMS-ESI (m/z): [M+H]⁺ Calcd for C46H₇₉NO₁₀Na⁺828.5596; Found 828.5577; [M−Na⁺2H]⁺ Calcd for C46H₈₀NO₁₀⁺806.5777; Found 806.5772.

### Example 1.4: Preparation of Compound (4)

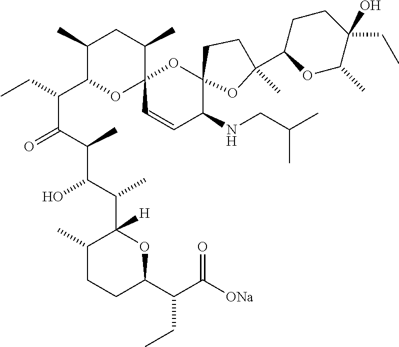

Yield: 40 mg, 74%. Isolated as a white amorphous solid, >95% pure by NMR and a single spot by TLC.

R_{f}: 0.50 in 66% EtOAc/n-hexane. Strains green with PMA.

¹H NMR (400 MHZ, CD₂Cl₂) δ 6.35 (dd, J=10.7, 5.6 Hz, ¹H), 6.15 (d, J=10.8 Hz, ¹H), 4.33 (q, J=6.8 Hz, ¹H), 4.16 (d, J=9.4 Hz, ¹H), 3.81 (dd, J=11.1, 4.9 Hz, ¹H), 3.72-3.60 (m, 2H), 3.41 (dd, J=12.0, 2.1 Hz, ¹H), 2.85 (d, J=5.6 Hz, ¹H), 2.82-2.73 (m, 2H), 2.72-2.65 (m, ¹H), 2.62 (dd, J=11.3, 6.7 Hz, ¹H), 2.41 (dd, J=11.3, 6.6 Hz, ¹H), 2.15-2.09 (m, 2H), 2.05-0.50 (m, 62H) ppm.

¹³C NMR (101 MHZ, CD₂Cl₂) δ 218.3, 184.1, 130.0, 122.5, 110.2, 99.7, 89.2, 76.4, 76.3, 75.5, 75.0, 71.8, 70.2, 67.7, 56.7, 56.0, 56.0, 51.6, 50.8, 40.8, 39.4, 37.6, 36.5, 33.4, 33.2, 33.0, 29.8, 29.7, 28.6, 28.3, 27.4, 24.2, 21.3, 20.8, 20.7, 20.5, 17.9, 17.4, 16.5, 15.1, 13.5, 12.8, 12.4, 11.0, 7.0, 6.8 ppm.

FT-IR (KBr): 3289 (br, m), 2960 (s), 2931 (s), 2874 (m), 1713 (s), 1660 (w), 1568 (s), 1459 (s), 1407 (m) cm-1.

HRMS-ESI (m/z): [M+H]⁺ Calcd for C46H₇₉NO₁₀Na⁺828.5596; Found 828.5578; [M−Na⁺2H]⁺ Calcd for C46H₈₀NO₁₀⁺806.5777; Found 806.5771.

### Example 1.5: Preparation of Compound (5)

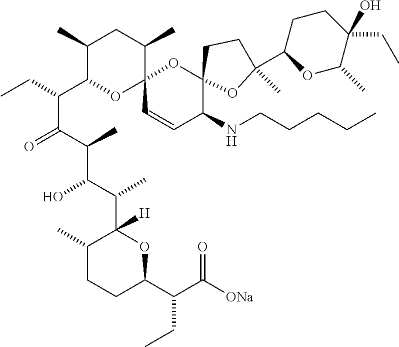

Yield: 45 mg, 82%. Isolated as a white amorphous solid, >95% pure by NMR and a single spot by TLC.

R_{f}: 0.30 in 66% EtOAc/n-hexane. Strains green with PMA.

¹H NMR (400 MHZ, CD₂Cl₂) δ 6.23 (dd, J=10.7, 5.6 Hz, ¹H), 6.03 (d, J=10.8 Hz, ¹H), 4.21 (q, J=6.7 Hz, ¹H), 4.04 (d, J=10.4 Hz, ¹H), 3.70 (dd, J=11.1, 4.8 Hz, ¹H), 3.62-3.49 (m, 2H), 3.29 (dd, J=11.9, 2.1 Hz, ¹H), 2.76 (d, J=5.6 Hz, ¹H), 2.71-2.60 (m, ¹H), 2.60-2.53 (m, 3H), 2.48 (ddd, J=11.3, 7.5, 6.4 Hz, ¹H), 2.02-1.96 (m, 2H), 1.90-0.50 (m, 64H) ppm.

¹³C NMR (101 MHZ, CD₂Cl₂) δ 218.4, 184.1, 130.0, 122.6, 110.1, 99.7, 89.2, 76.5, 76.3, 75.5, 75.1, 71.8, 70.2, 67.7, 56.1, 55.8, 51.6, 50.9, 48.6, 40.7, 39.5, 37.5, 36.5, 33.4, 33.2, 33.0, 31.1, 29.9, 29.8, 28.6, 28.3, 27.5, 24.2, 23.1, 21.4, 20.6, 17.9, 17.4, 16.6, 15.1, 14.4, 13.5, 12.8, 12.4, 11.0, 7.0, 6.8 ppm.

FT-IR (KBr): 3292 (br, m), 2961 (s), 2932 (s), 2873 (m), 1713 (s), 1660 (w), 1567 (s), 1459 (s), 1406 (m) cm-1.

HRMS-ESI (m/z): [M+H]⁺ Calcd for C₄₇H₈₁NO₁₀Na⁺842.5753; Found 842.5728; [M−Na⁺2H]⁺ Calcd for C47H₈₂NO₁₀⁺820.5933; Found 820.5925.

### Example 1.6: Preparation of Compound (6)

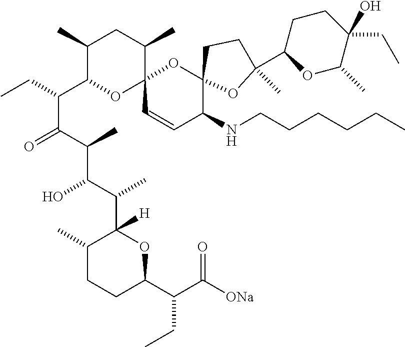

Yield: 37 mg, 70%. Isolated as a white amorphous solid, >95% pure by NMR and a single spot by TLC.

R_{f}: 0.31 in 66% EtOAc/n-hexane. Strains green with PMA.

¹H NMR (400 MHZ, CD₂Cl₂) δ 6.31 (dd, J=10.7, 5.6 Hz, ¹H), 6.11 (d, J=10.7 Hz, ¹H), 4.29 (q, J=6.7 Hz, ¹H), 4.12 (d, J=10.3 Hz, ¹H), 3.78 (dd, J=11.0, 4.9 Hz, ¹H), 3.64 (dd, J=10.0, 1.7 Hz, ¹H), 3.60 (d, J=10.1 Hz, ¹H), 3.37 (dd, J=11.9, 1.7 Hz, ¹H), 2.84 (d, J=5.6 Hz, ¹H), 2.79-2.69 (m, 3H), 2.68-2.62 (m, ¹H), 2.56 (dt, J=11.3, 6.8 Hz, ¹H), 2.08-2.02 (m, 2H), 2.00-0.50 (m, 66H) ppm.

¹³C NMR (101 MHZ, CD₂Cl₂) δ 217.8, 183.5, 129.4, 122.0, 109.5, 99.1, 88.6, 75.9, 75.7, 74.9, 74.5, 71.2, 69.6, 67.1, 55.5, 55.2, 51.0, 50.3, 48.0, 40.1, 38.9, 36.9, 35.9, 32.8, 32.6, 32.4, 31.7, 30.7, 29.2, 28.0, 27.7, 26.9, 26.8, 23.6, 22.6, 20.8, 20.0, 17.3, 16.8, 16.0, 14.5, 13.8, 12.9, 12.3, 11.8, 10.4, 6.5, 6.20 ppm.

FT-IR (KBr): 3297 (br, m), 2958 (s), 2930 (s), 2872 (m), 2858 (m), 1714 (s), 1668 (w), 1565 (s), 1459 (s), 1406 (m) cm-1.

HRMS-ESI (m/z): [M+H]⁺ Calcd for C₄₈H₈₃NO₁₀Na⁺856.5909; Found 856.5889; [M−Na⁺2H]⁺ Calcd for C₄₈H₈₄NO₁₀⁺834.6090; Found 834.6084.

### Example 1.7: Preparation of Compound (7)

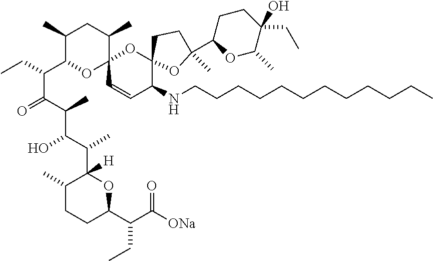

Yield: 60 mg, 85%. Isolated as a white amorphous solid, >95% pure by NMR and a single spot by TLC.

R_{f}: 0.40 in 66% EtOAc/n-hexane. Strains green with PMA.

¹H NMR (400 MHZ, CD₂Cl₂) δ 6.31 (dd, J=10.7, 5.6 Hz, ¹H), 6.11 (d, J=10.8 Hz, ¹H), 4.28 (dd, J=13.5, 6.7 Hz, ¹H), 4.12 (d, J=10.4 Hz, ¹H), 3.77 (dd, J=11.1, 4.7 Hz, ¹H), 3.64 (dd, J=10.1, 2.0 Hz, ¹H), 3.59 (d, J=10.2 Hz, ¹H), 3.37 (dd, J=11.9, 2.1 Hz, ¹H), 2.83 (d, J=5.6 Hz, ¹H), 2.79-2.73 (m, ¹H), 2.74-2.68 (m, 2H), 2.68-2.61 (m, ¹H), 2.55 (ddd, J=11.3, 7.4, 6.3 Hz, ¹H), 2.10-2.03 (m, 2H), 2.00-0.50 (m, 78H) ppm.

¹³C NMR (101 MHZ, CD₂Cl₂) δ 218.1, 183.9, 129.8, 122.4, 109.9, 99.5, 89.0, 76.2, 76.0, 75.3, 74.8, 71.6, 70.0, 67.4, 55.8, 55.5, 51.4, 50.6, 48.3, 40.5, 39.2, 37.3, 36.3, 33.2, 32.9, 32.8, 32.3, 31.2, 30.03, 30.01, 30.00, 29.9, 29.7, 29.6, 28.4, 28.1, 27.5, 27.2, 24.0, 23.1, 21.1, 20.3, 17.7, 17.2, 16.3, 14.9, 14.3, 13.3, 12.6, 12.2, 10.8, 6.8, 6.6 ppm, one signal overlapped. FT-IR (KBr): 3288 (br, m), 2960 (s), 2927 (s), 2854 (m), 1713 (s), 1568 (s), 1459 (s), 1406 (m) cm-1.

HRMS-ESI (m/z): [M+H]⁺ Calcd for C₅₄H₉₅NO₁₀Na⁺940.6848; Found 940.6829; [M−Na⁺2H]⁺

Calcd for C₅₄H₉₆NO₁₀⁺918.7029; Found 918.7024.

### Example 1.8: Preparation of Compound (8)

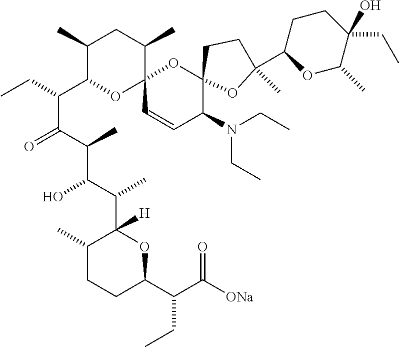

Yield: 41 mg, 76%. Isolated as a white amorphous solid, >95% pure by NMR and a single spot by TLC.

R_{f}: 0.42 in 33% EtOAc/n-hexane. Strains green with PMA.

¹H NMR (400 MHZ, CD₂Cl₂) δ 6.28 (dd, J=11.0, 1.6 Hz, ¹H), 5.98 (dd, J=11.0, 4.2 Hz, ¹H), 4.22 (q, J=6.7 Hz, ¹H), 4.11 (d, J=10.2 Hz, ¹H), 3.79 (dd, J=11.1, 4.8 Hz, ¹H), 3.63 (d, J=10.1 Hz, 2H), 3.38 (dd, J=12.0, 2.1 Hz, 2H), 2.75 (td, J=11.2, 3.3 Hz, ¹H), 2.69-2.64 (m, 2H), 2.56-2.43 (m, 3H), 2.31 (dq, J=13.4, 6.7 Hz, 2H), 2.20-0.50 (m, 61H) ppm.

¹³C NMR (101 MHZ, CD₂Cl₂) δ 218.4, 183.7, 125.6, 123.9, 110.9, 98.1, 87.3, 76.1, 75.8, 75.1, 74.9, 71.2, 69.5, 67.1, 56.4, 55.7, 51.0, 50.1, 44.4, 40.4, 38.6, 36.7, 35.9, 33.0, 32.6, 32.4, 29.8, 28.0, 27.6, 26.9, 23.6, 21.1, 19.9, 17.3, 16.8, 15.7, 14.6, 13.7, 12.8, 12.2, 12.0, 10.4, 6.5, 6.2 ppm.

FT-IR (KBr): 3300 (br, m), 2962 (s), 2933 (s), 2874 (m), 1714 (s), 1566 (s), 1458 (s), 1405 (m) cm-1.

HRMS-ESI (m/z): [M+H]⁺ Calcd for C₄₆H₇₉NO₁₀Na⁺828.5596; Found 828.5582; [M−Na⁺2H]⁺ Calcd for C₄₆H₈₀NO₁₀⁺806.5777; Found 806.5774.

### Example 1.9: Preparation of Compound (9)

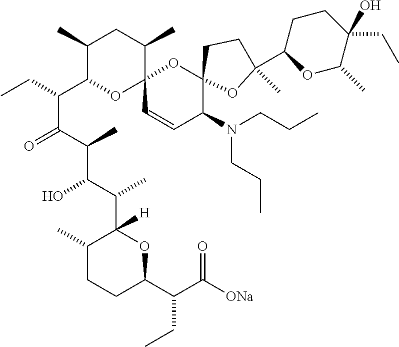

Yield: 45 mg, 79%. Isolated as a white amorphous solid, >95% pure by NMR and a single spot by TLC.

R_{f}: 0.57 in 33% EtOAc/n-hexane. Strains green with PMA.

¹H NMR (400 MHZ, CD₂Cl₂) δ 6.28 (dd, J=11.0, 1.7 Hz, ¹H), 6.00 (dd, J=11.0, 4.2 Hz, ¹H), 4.22 (q, J=6.8 Hz, ¹H), 4.11 (d, J=10.3 Hz, ¹H), 3.79 (dd, J=11.1, 4.8 Hz, ¹H), 3.68-3.59 (m, 2H), 3.37 (dd, J=12.0, 2.1 Hz, ¹H), 3.30 (dd, J=4.2, 1.8 Hz, ¹H), 2.77-2.70 (m, ¹H), 2.69-2.63 (m, 2H), 2.52-2.41 (m, ¹H), 2.40-2.25 (m, 4H), 2.00-0.50 (m, 65H) ppm.

¹³C NMR (101 MHz, CD₂Cl₂) δ 219.0, 184.2, 126.3, 124.4, 111.6, 98.7, 87.9, 76.7, 76.4, 75.6, 75.4, 71.8, 70.0, 67.7, 57.9, 56.3, 53.7, 51.6, 50.8, 41.1, 39.2, 37.3, 36.5, 33.6, 33.3, 33.0, 30.4, 28.6, 28.2, 27.5, 24.1, 22.2, 21.7, 20.5, 17.9, 17.5, 16.3, 15.2, 13.4, 12.8, 12.6, 12.1, 11.0, 7.1, 6.7 ppm.

FT-IR (KBr): 3297 (br, m), 2960 (s), 2933 (s), 2873 (m), 1714 (s), 1566 (s), 1459 (s), 1406 (m) cm-1.

HRMS-ESI (m/z): [M+H]⁺ Calcd for C₄₈H₈₃NO₁₀Na⁺856.5909; Found 856.5892; [M−Na⁺2H]⁺ Calcd for C₄₈H₈₄NO₁₀⁺834.6090; Found 834.6089.

### Example 1.10: Preparation of Compound (10)

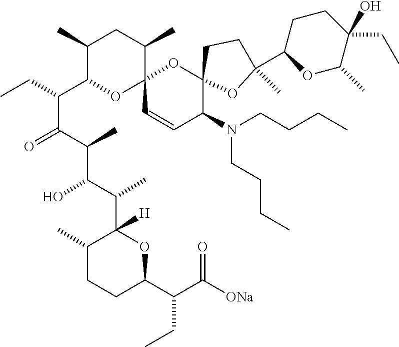

Yield: 30 mg, 55%. Isolated as a white amorphous solid, >95% pure by NMR and a single spot by TLC.

R_{f}: 0.59 in 33% EtOAc/n-hexane. Strains green with PMA.

¹H NMR (400 MHZ, CD₂Cl₂) δ 6.29 (dd, J=11.0, 1.7 Hz, ¹H), 6.01 (dd, J=11.0, 4.2 Hz, ¹H), 4.20 (q, J=6.7 Hz, ¹H), 4.11 (d, J=10.2 Hz, ¹H), 3.79 (dd, J=11.1, 4.8 Hz, ¹H), 3.66-3.60 (m, 2H), 3.37 (dd, J=12.0, 2.1 Hz, ¹H), 3.30 (dd, J=4.2, 1.7 Hz, ¹H), 2.77-2.64 (m, 3H), 2.51-2.35 (m, 3H), 2.34-2.25 (m, 2H), 2.00-0.50 (m, 69H) ppm.

¹³C NMR (101 MHZ, CD₂Cl₂) δ 218.9, 184.2, 126.1, 124.4, 111.6, 98.7, 87.9, 76.9, 76.4, 75.6, 75.3, 71.8, 69.9, 67.6, 57.7, 56.3, 51.6, 51.4, 50.8, 41.1, 39.2, 37.4, 36.5, 33.6, 33.3, 32.9, 31.5, 30.2, 28.6, 28.2, 27.5, 24.2, 21.7, 21.2, 20.5, 17.9, 17.5, 16.3, 15.2, 14.5, 13.4, 12.9, 12.6, 11.0, 7.1, 6.7 ppm.

FT-IR (KBr): 3300 (br, m), 2960 (s), 2933 (s), 2873 (m), 2860 (m), 1712 (s), 1566 (s), 1457 (s), 1406 (m) cm-1.

HRMS-ESI (m/z): [M+H]⁺ Calcd for C₅₀H₈₇NO₁₀Na⁺884.6222; Found 884.6212; [M−Na⁺2H]⁺ Calcd for C₅₀H₈₈NO₁₀⁺862.6403; Found 862.6410.

### Example 1.11: Preparation of Compound (11)

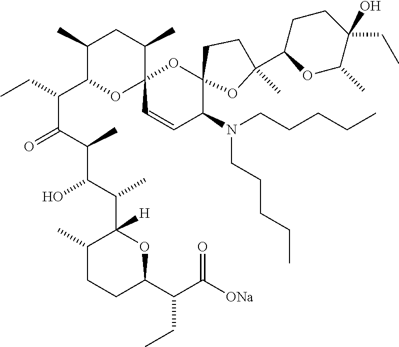

Yield: 35 mg, 60%. Isolated as a white amorphous solid, >95% pure by NMR and a single spot by TLC.

R_{f}: 0.62 in 33% EtOAc/n-hexane. Strains green with PMA.

¹H NMR (400 MHZ, CD₂Cl₂) δ 6.29 (dd, J=11.0, 1.6 Hz, ¹H), 6.01 (dd, J=11.0, 4.2 Hz, ¹H), 4.19 (q, J=6.7 Hz, ¹H), 4.11 (d, J=10.3 Hz, ¹H), 3.79 (dd, J=11.0, 4.7 Hz, ¹H), 3.69-3.60 (m, 2H), 3.37 (dd, J=12.0, 1.9 Hz, ¹H), 3.29 (dd, J=4.1, 1.6 Hz, ¹H), 2.70 (tdd, J=10.3, 9.3, 3.1 Hz, 3H), 2.45 (ddd, J=22.3, 13.0, 7.1 Hz, 2H), 2.39-2.25 (m, 3H), 2.00-0.50 (m, 73H) ppm.

¹³C NMR (101 MHZ, CD₂Cl₂) δ 218.9, 184.2, 126.1, 124.4, 111.6, 98.7, 87.9, 77.0, 76.4, 75.6, 75.3, 71.8, 69.9, 67.7, 57.8, 56.3, 51.7, 51.6, 50.8, 41.1, 39.2, 37.4, 36.5, 33.6, 33.3, 32.9, 30.3, 30.1, 29.0, 28.6, 28.2, 27.5, 24.2, 23.3, 21.7, 20.5, 17.9, 17.5, 16.3, 15.2, 14.5, 13.4, 12.9, 12.6, 11.0, 7.1, 6.8 ppm.

FT-IR (KBr): 3297 (br, m), 2959 (s), 2931 (s), 2872 (m), 2860 (m), 1714 (s), 1567 (s), 1459 (s), 1406 (m) cm-1.

HRMS-ESI (m/z): [M+H]⁺ Calcd for C₅₂H₉₁NO₁₀Na⁺912.6535; Found 912.6524; [M−Na⁺2H]⁺ Calcd for C₅₂H₉₂NO₁₀⁺890.6716; Found 890.6724.

### Example 1.12: Preparation of Compound (12)

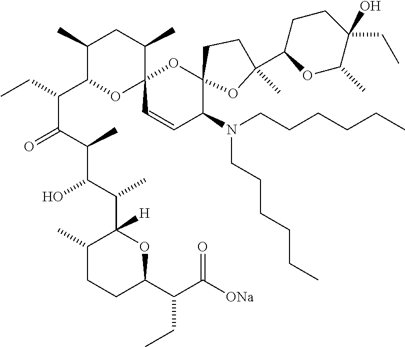

Yield: 50 mg, 82%. Isolated as a white amorphous solid, >95% pure by NMR and a single spot by TLC.

R_{f}: 0.64 in 33% EtOAc/n-hexane. Strains green with PMA.

¹H NMR (400 MHZ, CD₂Cl₂) δ 6.29 (dd, J=11.0, 1.6 Hz, ¹H), 6.01 (dd, J=11.0, 4.2 Hz, ¹H), 4.20 (q, J=6.7 Hz, ¹H), 4.12 (d, J=10.3 Hz, ¹H), 3.78 (dt, J=13.2, 6.5 Hz, ¹H), 3.68-3.59 (m, 2H), 3.38 (dd, J=12.0, 1.9 Hz, ¹H), 3.29 (dd, J=4.1, 1.6 Hz, ¹H), 2.72 (ddd, J=11.4, 8.5, 3.4 Hz, 2H), 2.68-2.63 (m, ¹H), 2.52-2.43 (m, ¹H), 2.43-2.36 (m, 2H), 2.36-2.27 (m, 2H), 2.00-0.50 (m, 77H) ppm.

¹³C NMR (101 MHZ, CD₂Cl₂) δ 218.9, 184.2, 126.2, 124.4, 111.6, 98.7, 87.9, 77.0, 76.4, 75.6, 75.3, 71.8, 69.9, 67.7, 57.8, 56.3, 51.7, 51.6, 50.8, 41.1, 39.2, 37.4, 36.5, 33.6, 33.3, 33.0, 32.5, 30.0, 29.3, 28.6, 28.2, 27.8, 27.5, 24.2, 23.4, 21.7, 20.5, 18.0, 17.6, 16.3, 15.2, 14.5, 13.5, 12.9, 12.6, 11.0 7.1, 6.8 ppm.

FT-IR (KBr): 3286 (br, m), 2960 (s), 2931 (s), 2873 (m), 2859 (m), 1713 (s), 1568 (s), 1459 (s), 1407 (m) cm-1.

HRMS-ESI (m/z): [M+H]⁺ Calcd for C₅₄H₉₅NO₁₀Na⁺940.6848; Found 940.6839; [M−Na⁺2H]⁺

Calcd for C₅₄H₉₆NO₁₀⁺918.7029; Found 918.7038.

### Example 1.13: Preparation of Compound (13)

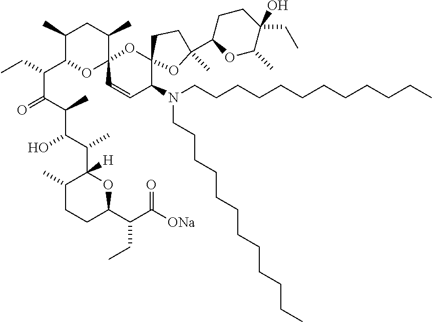

Yield: 21 mg, 45%. Isolated as a white amorphous solid, >95% pure by NMR and a single spot by TLC.

R_{f}: 0.79 in 33% EtOAc/n-hexane. Strains green with PMA.

¹H NMR (400 MHZ, CD₂Cl₂) δ 6.28 (dd, J=11.0, 1.6 Hz, ¹H), 6.00 (dd, J=11.0, 4.2 Hz, ¹H), 4.20 (q, J=6.6 Hz, ¹H), 4.11 (d, J=10.3 Hz, ¹H), 3.79 (dd, J=11.0, 4.6 Hz, ¹H), 3.71-3.59 (m, 2H), 3.38 (dd, J=12.0, 1.9 Hz, ¹H), 3.29 (dd, J=4.1, 1.5 Hz, ¹H), 2.75 (dd, J=11.1, 3.2 Hz, ¹H), 2.71-2.64 (m, 2H), 2.47 (dd, J=12.5, 3.6 Hz, ¹H), 2.43-2.38 (m, ¹H), 2.36 (d, J=8.1 Hz, ¹H), 2.34-2.25 (m, 2H), 2.00-0.50 (m, 101H) ppm.

¹³C NMR (101 MHZ, CD₂Cl₂) δ 218.9, 184.2, 126.1, 124.4, 111.6, 98.7, 87.9, 76.9, 76.4, 75.6, 75.3, 71.8, 69.9, 67.7, 57.7, 56.3, 51.7, 51.6, 50.8, 41.1, 39.2, 37.4, 36.5, 33.6, 33.3, 32.9, 32.1, 30.6, 30.4, 30.3, 30.24, 30.22, 30.0, 29.9, 29.3, 28.6, 28.2, 28.1, 27.5, 24.2, 23.3, 21.7, 20.5, 17.9, 17.6, 16.3, 15.2, 14.5, 13.4, 12.9, 12.6, 11.0, 7.1, 6.8 ppm.

FT-IR (KBr): 3295 (br, m), 2958 (s), 2926 (s), 2872 (m), 2854 (m), 1714 (s), 1566 (s), 1459 (s), 1405 (m) cm-1.

HRMS-ESI (m/z): [M+H]⁺ Calcd for C₆H₁₁₉NO₁₀Na⁺1108.8726; Found 1108.8715; [M−Na⁺2H]⁺ Calcd for C₆₆H₁₂₀NO₁₀⁺1086.8907; Found 1086.8920.

### Example 1.14: Preparation of Compound (14)

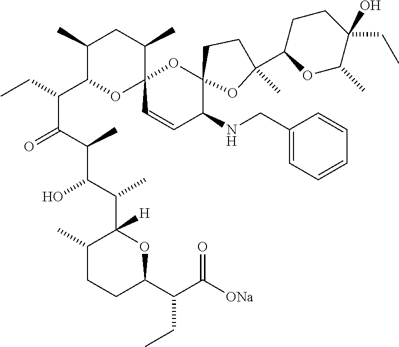

Yield: 48 mg, 75%. Isolated as a white amorphous solid, >95% pure by NMR and a single spot by TLC.

R_{f}: 0.65 in 50% EtOAc/n-hexane. Strains green with PMA.

¹H NMR (400 MHZ, CD₂Cl₂) δ 7.19 (d, J=4.4 Hz, 4H), 7.12 (dt, J=5.9, 4.2 Hz, ¹H), 6.28 (dd, J=10.7, 5.6 Hz, ¹H), 6.09 (d, J=10.7 Hz, ¹H), 4.18 (q, J=6.7 Hz, ¹H), 4.04 (d, J=10.4 Hz, ¹H), 3.83 (d, J=12.7 Hz, ¹H), 3.74-3.63 (m, 2H), 3.61-3.50 (m, 2H), 3.26 (dd, J=11.8, 1.8 Hz, ¹H), 2.90 (d, J=5.5 Hz, ¹H), 2.73-2.65 (m, ¹H), 2.64-2.54 (m, 2H), 2.00-0.50 (m, 57H) ppm.

¹³C NMR (101 MHZ, CD₂Cl₂) δ 218.4, 184.1, 141.5, 129.3, 128.8, 127.3, 123.1, 110.0, 99.7, 89.3, 76.5, 76.3, 75.5, 75.0, 71.8, 70.2, 67.7, 56.1, 55.1, 52.5, 51.6, 50.9, 40.7, 39.4, 37.6, 36.5, 33.4, 33.1, 33.0, 29.7, 28.6, 28.3, 27.4, 24.2, 21.3, 20.5, 17.8, 17.4, 16.5, 15.1, 13.5, 12.9, 12.4, 11.0, 7.0, 6.8 ppm.

FT-IR (KBr): 3317 (br, m), 2961 (s), 2933 (s), 2874 (m), 1713 (s), 1567 (s), 1495 (s), 1458 (s), 1406 (m) cm-1.

HRMS-ESI (m/z): [M+H]⁺ Calcd for C₄₉H₇₇NO₁₀Na⁺862.5440; Found 862.4532; [M−Na⁺2H]⁺ Calcd for C₄₉H₇₈NO₁₀⁺840.5620; Found 840.5628.

### Example 1.15: Preparation of Compound (15)

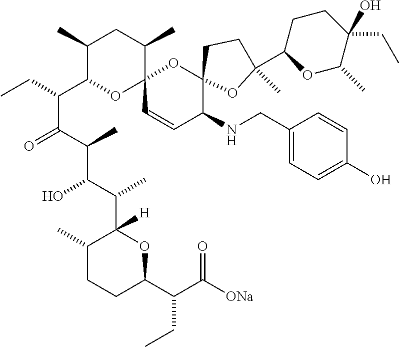

Yield: 43 mg, 76%. Isolated as a white amorphous solid, >95% pure by NMR and a single spot by TLC.

R_{f}: 0.60 in 50% EtOAc/n-hexane. Strains green with PMA.

¹H NMR (400 MHZ, CD₂Cl₂) δ 7.12 (td, J=7.9, 1.6 Hz, ¹H), 6.98 (d, J=6.3 Hz, ¹H), 6.77-6.72 (m, 2H), 6.43 (dd, J=10.7, 5.5 Hz, ¹H), 6.29 (d, J=10.8 Hz, ¹H), 4.29 (q, J=6.7 Hz, ¹H), 4.13 (d, J=10.5 Hz, ¹H), 3.77 (dd, J=11.1, 4.8 Hz, ¹H), 3.63 (dd, J=16.2, 6.0 Hz, 2H), 3.38 (dd, J=11.9, 1.9 Hz, ¹H), 3.10 (d, J=5.4 Hz, ¹H), 2.78 (dd, J=11.0, 2.9 Hz, ¹H), 2.73 (dd, J=10.8, 2.4 Hz ¹H), 2.66 (dd, J=10.2, 7.4 Hz, ¹H), 2.00-0.50 (m, 60H) ppm.

¹³C NMR (101 MHZ, CD₂Cl₂) δ 218.4, 184.3, 158.4, 129.4, 129.1, 127.7, 125.1, 123.5, 119.7, 116.7, 108.8, 99.8, 89.8, 76.5, 76.2, 75.8, 75.0, 71.8, 70.2, 67.8, 56.0, 55.1, 51.6, 51.2, 50.8, 40.6, 39.3, 37.8, 36.5, 33.4, 33.1, 32.9, 29.7, 28.6, 28.3, 27.4, 24.3, 21.4, 20.5, 17.8, 17.4, 16.5, 15.1, 13.5, 12.9, 12.4, 11.0, 7.0, 6.7 ppm.

FT-IR (KBr): 3433 (br, m), 3320 (br, m), 2953 (s), 2932 (s), 2872 (m), 1713 (s), 1563 (s), 1456 (s), 1428 (s), 1405 (m) cm-1.

HRMS-ESI (m/z): [M+H]⁺ Calcd for C₄₉H₇₇NO₁₀Na⁺878.5389; Found 878.5381; [M−Na⁺2H]⁺ Calcd for C₄₉H₇₉NO₁₀⁺856.5569; Found 856.5575.

### Example 1.16: Preparation of Compound (16)

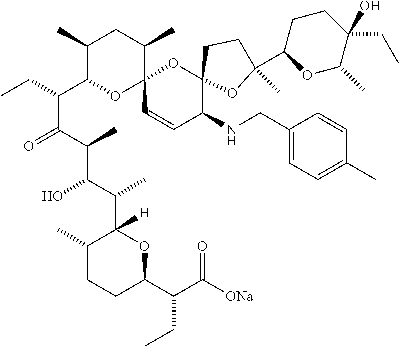

Yield: 51 mg, 89%. Isolated as a white amorphous solid, >95% pure by NMR and a single spot by TLC.

R_{f}: 0.63 in 50% EtOAc/n-hexane. Strains green with PMA.

¹H NMR (400 MHZ, CDCl₃) δ 7.15 (d, J=7.9 Hz, 2H), 7.08 (d, J=8.2 Hz, 2H), 6.49 (dd, J=10.7, 5.6 Hz, ¹H), 6.19 (d, J=10.8 Hz, ¹H), 4.39 (dd, J=13.8, 6.9 Hz, ¹H), 4.24 (d, J=10.3 Hz, ¹H), 3.90 (dd, J=11.0, 4.9 Hz, ¹H), 3.85 (d, J=12.5 Hz, ¹H), 3.72 (dd, J=15.5, 7.2 Hz, 2H), 3.56 (d, J=10.0 Hz, ¹H), 3.34-3.29 (m, ¹H), 3.03 (d, J=5.7 Hz, ¹H), 2.87 (td, J=11.0, 3.3 Hz, ¹H), 2.68 (dd, J=11.0, 2.6 Hz, ¹H), 2.64-2.59 (m, ¹H), 2.31 (s, 3H), 2.20-0.50 (m, 57H) ppm.

¹³C NMR (101 MHZ, CDCl₃) δ 216.8, 184.1, 137.7, 136.3, 129.0, 129.0, 128.2, 122.6, 109.3, 99.1, 88.7, 75.7, 75.6, 74.9, 74.5, 71.5, 69.8, 67.1, 55.2, 54.6, 51.6, 51.2, 50.4, 40.1, 38.9, 37.2, 36.0, 32.9, 32.5, 32.4, 29.7, 29.0, 27.9, 26.9, 23.9, 21.1, 20.8, 19.9, 17.5, 17.0, 16.0, 14.6, 13.2, 12.5, 11.8, 10.6, 6.7, 6.5 ppm.

FT-IR (KBr): 3295 (br, m), 2961 (s), 2931 (s), 2874 (m), 1713 (s), 1567 (s), 1458 (s), 1406 (m) cm-1.

HRMS-ESI (m/z): [M+H]⁺ Calcd for C₅₀H₇₉NO₁₁Na⁺876.5596; Found 876.5593; [M−Na⁺2H]⁺ Calcd for C₅₀H₈₀NO₁₁₊854.5777; Found 854.5777.

### Example 1.17: Preparation of Compound (17)

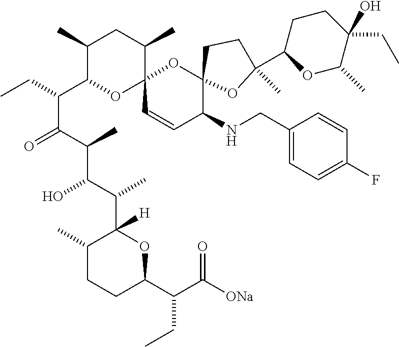

Yield: 70 mg, 88%. Isolated as a white amorphous solid, >95% pure by NMR and a single spot by TLC.

R_{f}: 0.67 in 50% EtOAc/n-hexane. Strains green with PMA.

¹H NMR (400 MHZ, CDCl₃) 0 7.22 (dd, J=8.5, 5.6 Hz, 2H), 6.95 (dd, J=12.0, 5.3 Hz, 2H), 6.48 (dd, J=10.7, 5.6 Hz, ¹H), 6.20 (d, J=10.8 Hz, ¹H), 4.37 (q, J=6.6 Hz, ¹H), 4.23 (d, J=10.3 Hz, ¹H), 3.89 (dd, J=11.1, 4.9 Hz, ¹H), 3.85 (d, J=11.9 Hz, ¹H), 3.74-3.68 (m, ¹H), 3.56 (d, J=10.1 Hz, ¹H), 3.30 (dd, J=11.9, 1.9 Hz, ¹H), 3.00 (d, J=5.6 Hz, ¹H), 2.87 (td, J=10.9, 3.1 Hz, ¹H), 2.67 (dt, J=7.1, 3.6 Hz, ¹H), 2.62 (dd, J=10.2, 7.4 Hz, ¹H), 2.20-0.50 (m, 58H) ppm.

¹³C NMR (101 MHZ, CDCl₃) δ 216.8, 184.1, 162.8, 160.9, 136.4, 129.9, 129.9, 128.8, 122.8, 115.2, 115.0, 109.3, 99.1, 88.8, 75.7, 75.7, 74.9, 74.5, 71.6, 69.8, 67.2, 55.3, 54.3, 51.3, 51.0, 50.4, 40.1, 38.9, 37.3, 36.0, 34.1, 32.9, 32.5, 32.4, 29.0, 27.9, 26.9, 24.0, 22.3, 20.9, 20.0, 17.5, 17.0, 16.1, 14.6, 14.1, 13.2, 12.5, 11.8, 10.6, 6.7, 6.5 ppm.

FT-IR (KBr): 3321 (br, m), 2962 (s), 2932 (s), 2874 (m), 1713 (s), 1567 (s), 1510 (s), 1459 (s), 1406 (m) cm⁻¹.

HRMS-ESI (m/z): [M+H]⁺ Calcd for C₄₉H₇₆FNO₁₀Na⁺880.5345; Found 880.5337; [M−Na⁺2H]⁺ Calcd for C₄₉H₇₇FNO₁₀⁺858.5526; Found 858.5532.

### Example 1.18: Preparation of Compound (18)

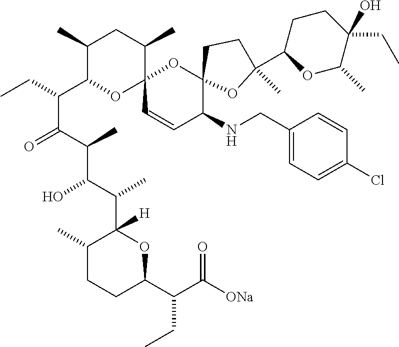

Yield: 34 mg, 58%. Isolated as a white amorphous solid, >95% pure by NMR and a single spot by TLC.

R_{f}: 0.58 in 50% EtOAc/n-hexane. Strains green with PMA.

¹H NMR (400 MHZ, CDCl₃) δ 7.18 (d, J=8.8 Hz, 2H), 7.13 (d, J=8.5 Hz, 2H), 6.42 (dd, J=10.7, 5.6 Hz, ¹H), 6.14 (d, J=10.8 Hz, ¹H), 4.30 (q, J=6.7 Hz, ¹H), 4.17 (d, J=10.2 Hz, ¹H), 3.83 (dd, J=10.6, 4.2 Hz, ¹H), 3.78 (d, J=12.9 Hz, ¹H), 3.68-3.61 (m, 2H), 3.49 (d, J=10.1 Hz, ¹H), 3.23 (d, J=11.6 Hz, ¹H), 2.93 (d, J=5.6 Hz, ¹H), 2.80 (td, J=10.9, 3.1 Hz, ¹H), 2.61 (dd, J=11.0, 2.4 Hz, ¹H), 2.55 (dd, J=10.2, 7.4 Hz, ¹H), 2.20-0.50 (m, 57H) ppm.

¹³C NMR (101 MHZ, CDCl₃) δ 216.8, 184.2, 139.1, 132.5, 129.7, 128.7, 128.4, 122.8, 109.2, 99.0, 88.8, 75.7, 75.6, 74.9, 74.5, 71.5, 69.8, 67.1, 55.2, 54.3, 51.2, 51.0, 50.4, 40.1, 38.8, 37.3, 36.0, 32.9, 32.5, 32.4, 29.7, 28.9, 27.9, 27.9, 26.9, 23.9, 20.8, 19.9, 17.5, 17.0, 16.0, 14.6, 13.2, 12.5, 11.8, 10.6, 6.7, 6.5 ppm.

FT-IR (KBr): 3322 (br, m), 2962 (s), 2932 (s), 2874 (m), 1713 (s), 1663 (s), 1567 (s), 1492 (s), 1459 (s), 1407 (m) cm-1.

HRMS-ESI (m/z): [M+H]⁺ Calcd for C₄₉H₇₆CINO₁₀Na⁺896.5050; Found 869.5046; [M−Na⁺2H]⁺ Calcd for C₄₉H₇₇CINO₁₀₊874.5231; Found 874.5238.

### Example 2: Preparation of C₂₀-epi-diironomycin

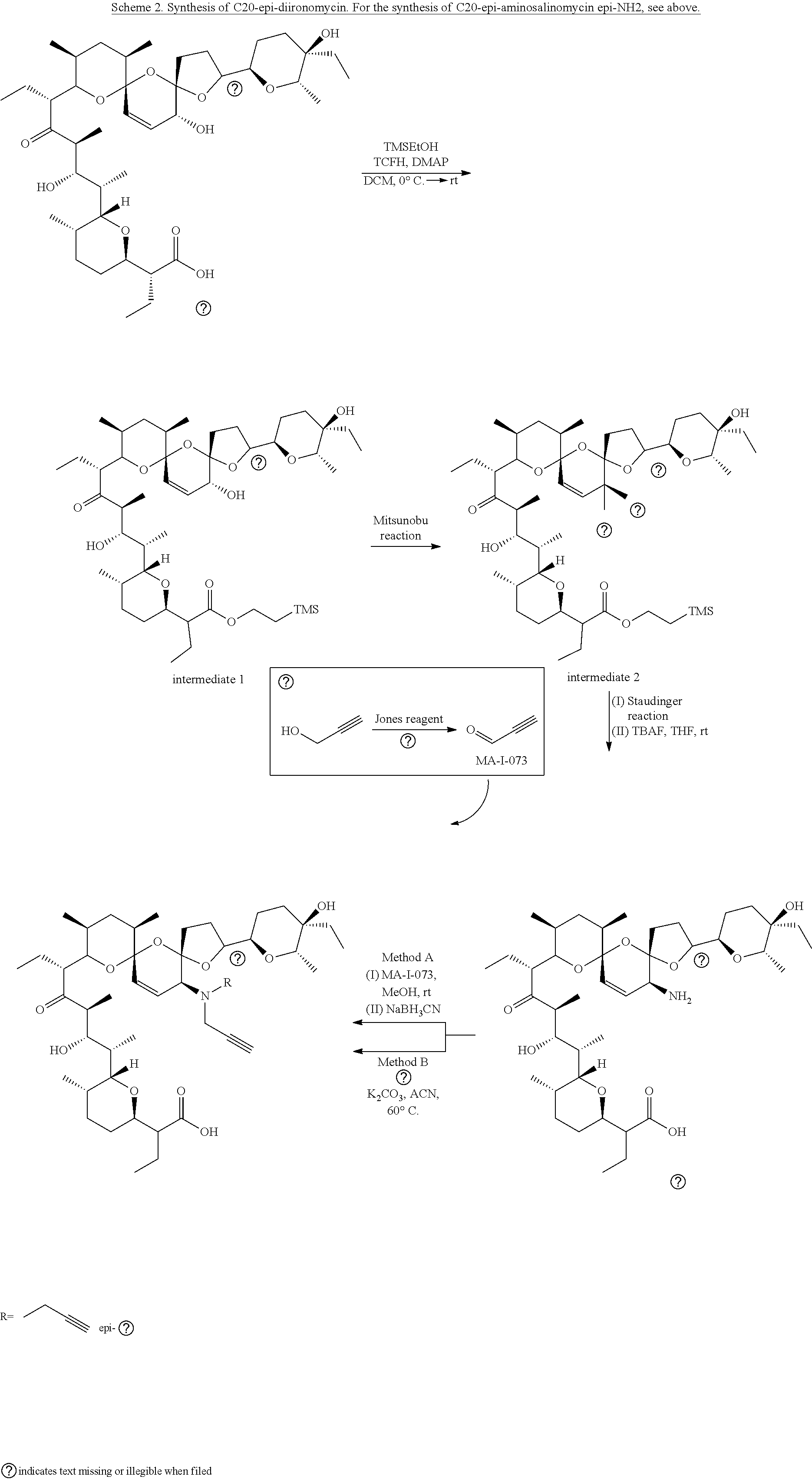

### Synthesis of C₂₀-epi-diironomycin

Method A: To a stirred solution of C₂₀-epi-aminosalinomycin (50 mg, 0.07 mmol, 1.0 equiv.) in anhydrous MeOH (2 mL) at room temperature, propynal (MA-I-073, freshly prepared) (8.8 mg, 0.16 mmol, 2.2 equiv.) and glacial acetic acid (2 drops) were added, and the resulting mixture was stirred for 4 h. After that, NaBH₃CN (9.0 mg, 0.15 mmol, 2.0 equiv.) dissolved in anhydrous MeOH (1 mL) was introduced dropwise. The mixture was stirred overnight, then the solvent was evaporated in vacuo to dryness. The residue was purified using HPLC equipped with a C18-reverse phase column (gradient: ACN/H₂O 1/1 to ACN). Pure product of the reaction was obtained as a white amorphous solid.

Method B: To a stirred solution of C20-epi-aminosalinomycin (50 mg, 0.07 mmol, 1.0 equiv.) and K₂CO₃ (10 mg, 0.07 mmol, 1.0 equiv.) in anhydrous ACN (2 mL) at room temperature, propargyl bromide (80 wt % in toluene) (28 mg, 0.24 mmol, 3.5 equiv.) was added drop by drop. The reaction mixture was then heated to 60° C., and stirred vigorously for the next 20 h. After that, the reaction mixture was filtered and the filtrate was evaporated in vacuo. The oily residue was dissolved in CH₂Cl₂ (10 mL), and extracted twice with aqueous solution of H₂SO₄ (15 mM, 2×7 mL) and water (7 mL). The organic layer was evaporated under reduced pressure, and purified chromatographically using HPLC equipped with a C18-reverse phase column (gradient: ACN/H₂O 1/1 to ACN). Pure product of the reaction was obtained as a white amorphous solid.

C20-epi-dipropargyloaminosalinomycin epi-diAM5 (compound 19): Yield: 1.4 mg, 3%. Isolated as a white amorphous solid, >95% pure by NMR, and a single spot by TLC; R_{f}: 0.70 in CH₂Cl₂/EtOAc 50%. Strain green with PMA; ¹H NMR (500 MHZ, CD₂Cl₂) 0 6.10 (dd, J=10.4, 1.6 Hz, ¹H), 5.54 (dd, J=10.4, 2.9 Hz, ¹H), 4.01 (dd, J=8.7, 8.2 Hz, 2H), 3.78 (ddd, J=17.5, 15.3, 6.1 Hz, 4H), 3.70-3.60 (m, 3H), 3.57 (dd, J=11.2, 1.3 Hz, ¹H), 3.46-3.40 (m, ¹H), 3.14-2.92 (m, 3H), 2.85 (dd, J=8.5, 4.3 Hz, ¹H), 2.77 (dd, J=11.3, 7.2 Hz, ¹H), 2.34-2.26 (m, 2H), 2.16 (dd, J=11.3, 8.6 Hz, ¹H), 2.10-0.60 (m, 53H) ppm; ¹³C NMR (126 MHZ, CD₂Cl₂) δ 217.6, 177.2, 130.9, 127.2, 109.9, 99.0, 85.3, 81.2, 78.2, 76.6, 75.3, 72.5, 72.1 (2C), 71.7, 71.3, 71.1, 60.8, 56.4, 49.6, 48.3, 41.4, 39.5, 36.8, 36.1, 34.8, 33.2, 31.0, 29.7, 29.4, 28.7, 26.4, 22.8, 22.7, 21.2, 21.0, 20.2, 18.6, 16.2, 14.6, 13.9, 13.5, 12.1, 11.5, 7.4, 6.5 ppm, two signals overlapped; HRMS (ESI+) m/z [M+H]⁺ Calcd for C₄₈H₇₆NO₁₀ 826.5469, Found 826.5467.

### Example 3: IC₅₀ of the compounds of the invention

The compounds have been evaluated against a model of breast cancer stem cells (HMLER) and matching non-stem cells (namely ID2 of HIMLER CD₂₄^{high}/CD₄^{high/low}), and in MCF10A cells (normal breast cell line).

Table 1 below represents the corresponding results.

Selectivity was calculated as the ratio of the IC₅₀ ID2 to the IC₅₀ HMLER.

Each IC₅₀ value was determined in biological triplicate (three independent biological experiments), and each triplicate was determined in at least technical duplicate.

| | HMLER | ID2 | | |
|---|---|---|---|---|
| | cancer cells | cancer cells | Selec- | MCF10A |
| Compound | (IC₅₀, μM) | (IC₅₀, μM) | tivity | (IC₅₀, μM) |
| Salinomycin | 1.39 ± 0.10 | 4.36 ± 0.5 | 3.1 | 1.13 ± 0.14 |
| 1 (ET1) | 0.53 ± 0.08 | 3.25 ± 3.3 | 6.1 | 2.61 ± 0.78 |
| 2 (ET2) | 0.17 ± 0.02 | 1.71 ± 0.4 | 10.1 | 1.56 ± 0.68 |
| 3 (ET4) | 0.12 ± 0.02 | 1.83 ± 0.7 | 15.2 | 1.46 ± 0.08 |
| 4 (ET5) | 0.07 ± 0.05 | 0.77 ± 0.46 | 11.0 | 1.30 ± 0.46 |
| 5 (ET6) | 0.11 ± 0.03 | 1.19 ± 0.6 | 10.8 | 0.46 ± 0.27 |
| 6 (ET8) | 0.03 ± 0.01 | 0.74 ± 0.29 | 24.7 | 1.50 ± 0.50 |
| 7 (ET10) | 0.17 ± 0.04 | 0.69 ± 0.05 | 4.1 | n.d. |
| 8 (ET11) | 0.003 ± 0.0005 | 0.27 ± 0.04 | 90.0 | 1.39 ± 0.18 |
| 9 (ET12) | 0.009 ± 0.0002 | 0.18 ± 0.02 | 20.0 | 0.47 ± 0.19 |
| 10 (ET13) | 0.85 ± 0.22 | 6.48 ± 0.5 | 7.6 | n.d. |
| 11 (ET14) | 0.33 ± 0.12 | 2.53 ± 1.00 | 7.7 | 3.93 ± 0.29 |
| 12 (ET15) | 0.30 ± 0.16 | 3.98 ± 2.20 | 13.3 | 1.70 ± 0.44 |
| 13 (ET17) | 1.92 ± 0.50 | 10.02 ± 3.60 | 5.2 | n.d. |
| 14 (ET18) | 0.12 ± 0.003 | 0.75 ± 0.12 | 6.2 | 4.67 ± 0.73 |
| 15 (ET19) | 0.10 ± 0.01 | 1.24 ± 0.02 | 12.4 | 4.16 ± 0.57 |
| 16 (ET20) | 0.07 ± 0.04 | 0.89 ± 0.28 | 12.7 | 1.26 ± 0.18 |
| 17 (ET21) | 0.06 ± 0.01 | 0.61 ± 0.01 | 10.2 | 1.88 ± 0.11 |
| 18 (ET22) | 0.08 ± 0.02 | 0.88 ± 0.54 | 11.0 | 1.86 ± 0.31 |
| 19 (epi- | 2.400 | 8.433 | 3.5 | n.d. |
| diAM5) | | | | |

### Cell Culture

HMLER cells naturally repressing E-cadherin, obtained from human mammary epithelial cells infected with a retrovirus carrying hTERT, SV40, and the oncogenic allele H-rasV12, were cultured in DMEM/F12 (Gibco, 31331-028) supplemented with 10% FBS, 10 μg/mL insulin (Sigma-Aldrich, 10516), 0.5 ug/mL hydrocortisone (Sigma-Aldrich, H₀₈₈₈), and 0.5 ug/mL puromycin (Life Technologies, A11138-02); cells were a generous gift from Alain Puisieux (INSERM). All cells were incubated at 37° C. with 5% CO₂. HMLER CD₄₄low/high cells stained with CD₂₄-APC and CD₄₄-PE antibodies were sorted by FACS using an Aria Ilu (BD Biosciences) to obtain isolated CD₂₄^{low}/CD₄₄^{high} and CD₂₄^{high}/CD₄₄^{low} cell populations. HMLER CD₂₄^{low}/CD₄₄^{high} cells were supplemented with 10 ng/ml human epidermal growth factor (EGF, Miltenyi Biotec, 130-093-750, 100 ng/ml), while HMLER CD₂₄^{high}/CD₄₄^{low} cells were grown without EGF. MCF10A cells (ATCC, CRL-10317) were cultured in DMEM/F12 supplemented with 10% horse serum (Invitrogen, 16050-122), 10 μg/mL insulin, 10 ng/ml EGF, 0.5 μg/mL hydrocortisone, 100 ng/ml cholera toxin (Sigma-Aldrich, C₈₀₅₂), and 1× PenStrep (Invitrogen, 15070-063).

### Cell Viability Assay (IC₅₀)

The cell viability assay was carried out by plating 1000 cells per well in 96-well plates. The cells were treated for 72 h in a range between 12 nM and 50 UM or 0.3 nM and 4 μM using serial dilutions following the manufacturer's protocol. Very briefly, the CellTiter-Blue reagent (G8081, Promega) was added to the wells after 72 h of treatment, and cells were incubated for 3 h before fluorescence intensities (λₑₓ=560/20 nm; λₑₘ=590/10 nm) were recorded using a PerkinElmer Wallac 1420 Victor2 microplate reader. The IC₅₀ cell viability curves were plotted using the Prism 8 software for the synthesized compounds against HMLER CD₂₄^{low}/CD₄₄^{high} and the isogenic cell line HMLER CD₂₄^{high}/CD₄₄^{low}.

All derivatives were assessed for their antiproliferative activity and selectivity toward a well-established model of mesenchymal CSCs (HMLER CD₂₄^{low}/CD₄₄^{high}) together with their epithelial counterparts (HMLER CD₂₄^{high}/CD₄₄^{low}) lacking CSC properties. Most of these derivatives were found to be more potent and more selective against the mesenchymal state compared to the references Sal and ironomycin. Specifically, compounds 6, 8, and 9 were identified to be particularly interesting in this context, with IC₅₀ values of 30, 3, and 9 nM, respectively, together with their outstanding selectivities (Sls between 20.0 and 90.0). Concerning a structure-activity relationship (SAR), we found the following: (i) monosubstituted C₂₀-epi-amino derivatives of Sal are essentially more active than their corresponding disubstituted counterparts; (ii) with respect to secondary amine products, n-pentyl and n-hexyl substituents are more potent against the mesenchymal state, (iii) regarding tertiary amine derivatives, elongation of the aliphatic chains results in a decrease of the antiproliferative activity; and (iv) the introduction of the nonpolar or polar substituent at the para-position of the benzyl motif increases the selectivity.

1. A compound having the formula (I): 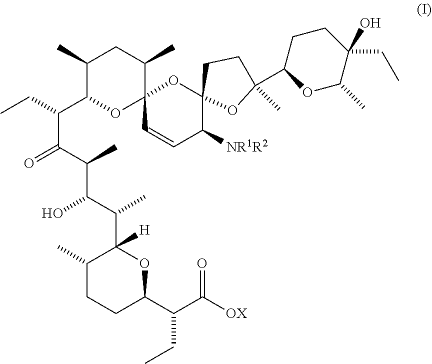
wherein:
R¹ and R² are identical or different and independently chosen from the group consisting of: H, a (C₁-C₂₀) alkyl group, a (C₃-C₆) cycloalkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, hydroxyl, amino, thiol (C₁-C₆) alkoxy, (C₁-C₆) alkylthio, (C₁-C₆) alkylamino, halo (C₁-C₆) alkyl, carboxyl and carboxy (C₁-C₆) alkyl;
X is H, K or Na,
with the proviso that at least one of R¹ and R² is other than H, and
the following compound being excluded:

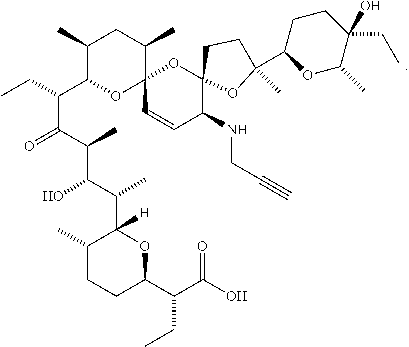 [SMILES: C#CCN[C@H]1C=C[C@]2(O[C@H]([C@@H](CC)C(=O)[C@@H](C)[C@@H](O)[C@H](C)[C@@H]3O[C@@H]([C@@H](CC)C(=O)O)CC[C@@H]3C)[C@@H](C)C[C@H]2C)O[C@@]12CC[C@@](C)([C@H]1CC[C@](O)(CC)[C@H](C)O1)O2]

2. The compound of claim 1, wherein R¹ and R² are different.

3. The compound of claim 1, wherein R¹ is selected from the group consisting of: a (C₁-C₂₀) alkyl group, a (C₃-C₆) cycloalkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, hydroxyl, amino, thiol (C₁-C₆) alkoxy, (C₁-C₆) alkylthio, (C₁-C₆) alkylamino, halo (C₁-C₆) alkyl, carboxyl and carboxy (C₁-C₆) alkyl, and R² is H.

4. The compound of claim 1, wherein R¹ is selected from the group consisting of: a (C₁-C₂₀) alkyl group, a (C₂-C₂₀) alkynyl group, and a (C₆-C₁₀) aryl (C₁-C₆) alkyl group, said (C₆-C₁₀) aryl being optionally substituted with at least substituent selected from the group consisting of: (C₁-C₆) alkyl, halogen, and hydroxyl.

5. The compound of claim 1, wherein R¹ is a linear (C₂-C₁₂) alkyl group, a propyn-1-yl group, or a benzyl group, optionally substituted with OH, methyl or halogen, preferably in para position.

6. The compound of claim 1, wherein R¹ and R² are identical, and preferably chosen from the group consisting of: a (C₁-C₂₀) alkyl group, preferably a linear (C₂-C₁₂) alkyl group, and a (C₂-C₂₀) alkynyl group, preferably a propynyl group.

7. The compound of claim 1, being chosen from the following compounds:

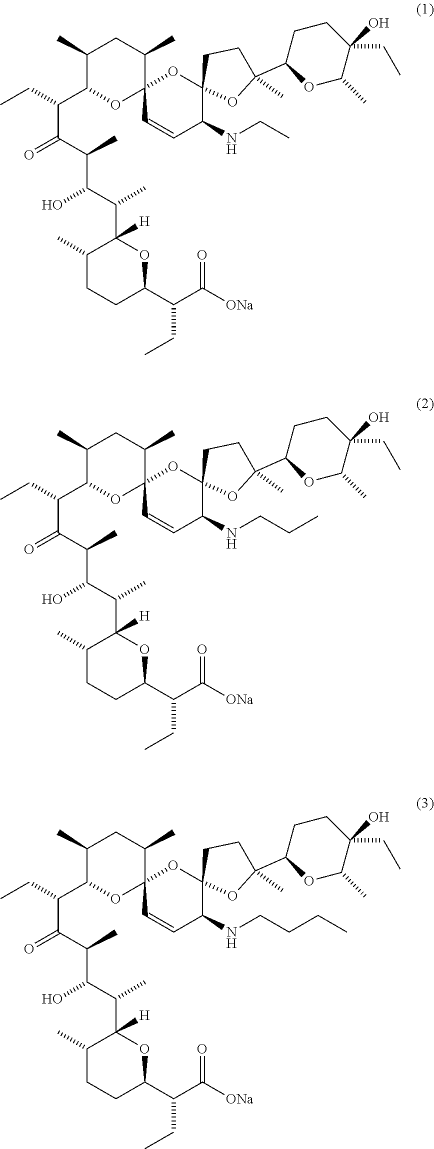

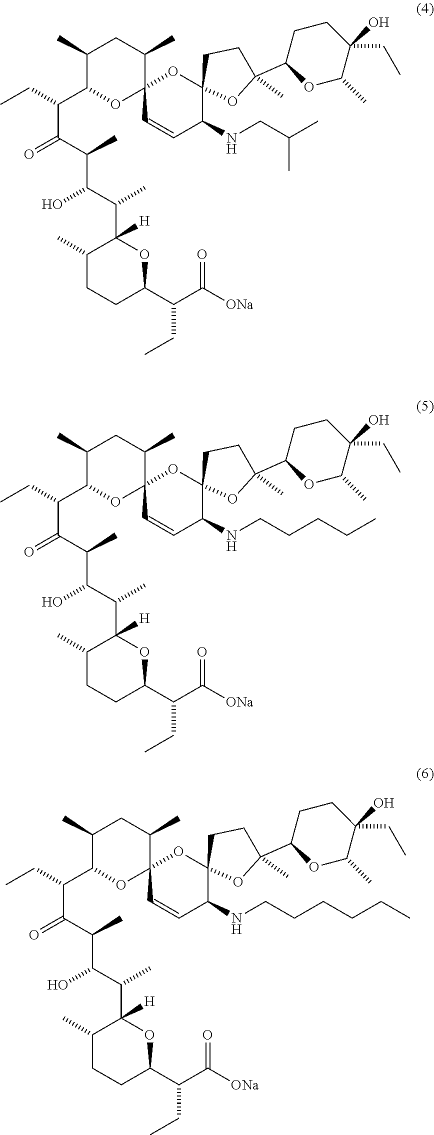

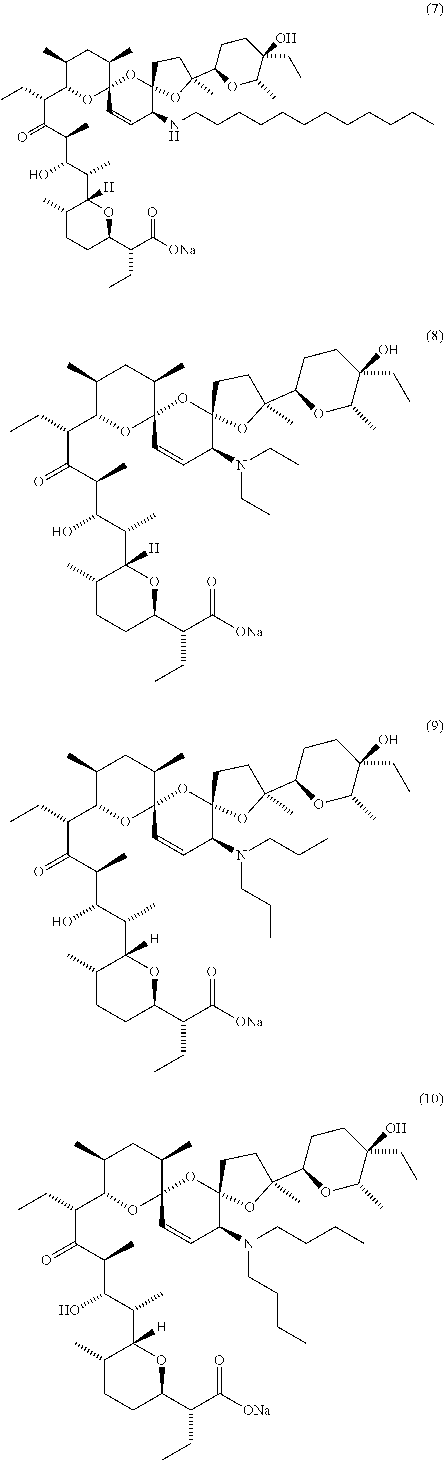

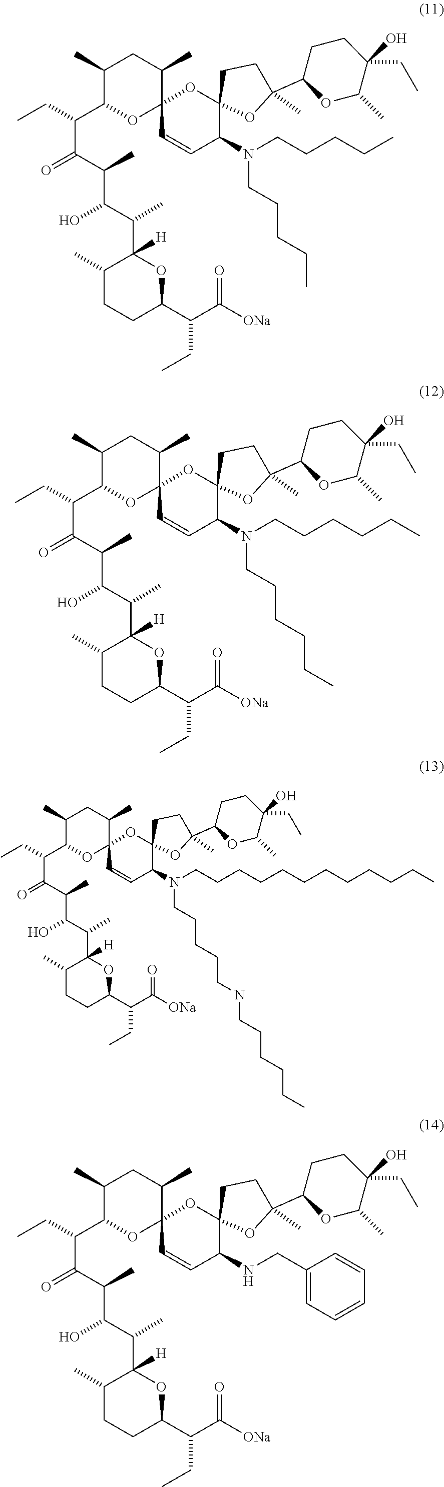

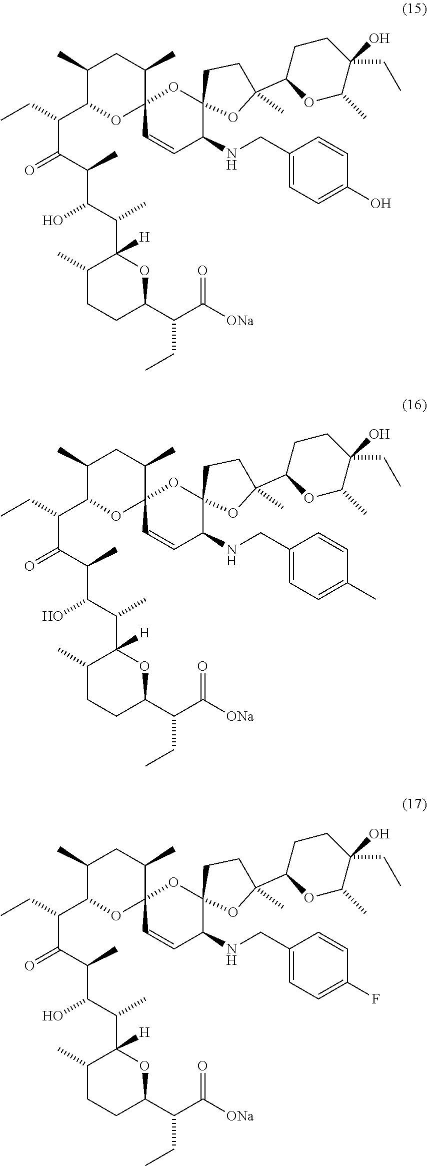

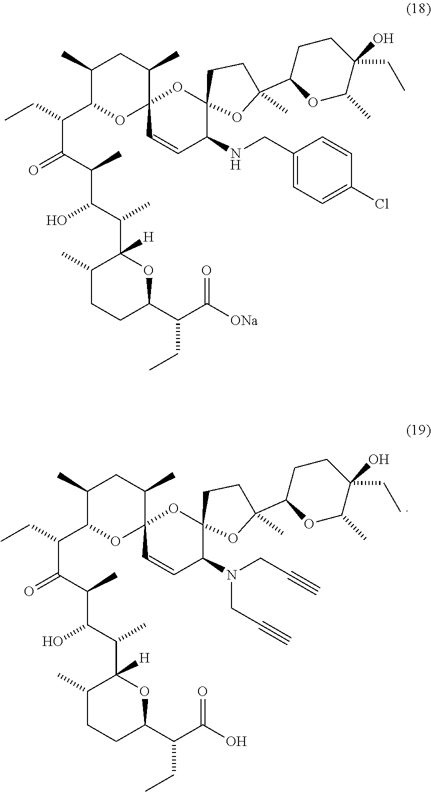

8. A process for the preparation of a compound according to claim 1, comprising the following steps:
the addition of an aldehyde R¹CHO, R¹ being as defined in formula (I), to a solution of a sodium salt of C20-epi-aminosalinomycin in a solvent, in order to obtain the corresponding imine, followed by the reduction of said imine into the corresponding amine compound of formula (I), or
the reaction of a bromide compound R¹Br, R¹ being as defined in formula (I), with the compound C20-epi-aminosalinomycin, preferably in a solvent.

9. (canceled)

10. A medicament comprising a compound according to claim 1, or a pharmaceutically acceptable salt thereof, and optionally at least one pharmaceutically acceptable excipient.

11. (canceled)

12. A medicament according to claim 10, wherein said medicament is a medicament for use for preventing and/or treating cancer.

13. The medicament according to claim 9, wherein said medicament is a medicament for preventing and/or treating cancer, wherein the cancer is selected from the group consisting of: a colon cancer, a colorectal cancer, a melanoma, a bone cancer, a breast cancer, a thyroid cancer, a prostate cancer, an ovarian cancer, a lung cancer, a pancreatic cancer, a glioma, a cervical cancer, an endometrial cancer, a head and neck cancer, a liver cancer, a bladder cancer, a renal cancer, a skin cancer, a stomach cancer, a testis cancer, an urothelial cancer or an adrenocortical carcinoma, leukemia, lymphoma, and multiple myeloma.

14. The medicament according to claim 9, wherein said medicament is a medicament for preventing cancer metastasis and/or for preventing cancer recurrence and/or for decreasing resistance to a chemotherapy in a subject.

15. A product comprising:
a) a compound according to claim 1, and
b) at least one additional therapy,
as combination product for a simultaneous, separate or sequential use for treating cancer, and/or for preventing cancer metastasis, and/or for preventing cancer recurrence, and/or for decreasing resistance to the additional therapy b), in a subject, said subject being preferably a human suffering from a cancer and resistant to chemotherapy,
said additional therapy b) being preferably immunotherapy, chemotherapy and/or radiotherapy.

16. The medicament according to claim 9, wherein said medicament is a medicament for preventing cancer metastasis and/or for preventing cancer recurrence and/or for decreasing resistance to a chemotherapy in a subject, wherein the cancer is selected from solid and non-solid cancers, preferably from a colon cancer, a colorectal cancer such as colorectal cancers with a BRAF mutation especially BRAF V600E, a melanoma, a bone cancer, a breast cancer such as triple-negative breast cancer, a thyroid cancer, a prostate cancer, an ovarian cancer, a lung cancer, a pancreatic cancer, a glioma such as a glioblastoma, a cervical cancer, an endometrial cancer, a head and neck cancer, a liver cancer, a bladder cancer, a renal cancer, a skin cancer, a stomach cancer, a testis cancer, an urothelial cancer or an adrenocortical carcinoma, leukemia such as acute myeloid leukemia, lymphoma and multiple myeloma.

17. A process for preventing and/or treating a cancer of a patient in need thereof, said process comprising a step of administering to said patient a compound according to claim 1 or a pharmaceutically acceptable salt thereof.

18. A process for preventing and/or treating a cancer of a patient in need thereof, said process comprising a step of administering to said patient a medicament according to claim 10.

19. A process for preventing and/or treating a cancer of a patient in need thereof, said process comprising a step of administering to said patient a product according to claim 15.
